(19) United States
(12) Patent Application Publication
(10) Pub. No.: US 2026/0139006 A1
(43) Pub. Date: May 21, 2026

(54) CYCLIC PEPTIDE AS INHIBITORS OF FREE-LIVING AMOEBA

(71) Applicant: Purdue Research Foundation, West Lafayette, IN (US)

(72) Inventors: Elizabeth Ivy Parkinson, West Lafayette, IN (US); Gabriela Coy, Bogotá (CO); Chenyang Lu, West Lafayette, IN (US); Samantha Nelson, West Lafayette, IN (US); Christopher Aaron Rice, West Lafayette, IN (US)

(73) Assignee: Purdue Research Foundation, West Lafayette, IN (US)

(21) Appl. No.: 19/328,414

(22) Filed: Sep. 15, 2025

Related U.S. Application Data
(60) Provisional application No. 63/720,864, filed on Nov. 15, 2024.

(51) Int. Cl. C07K 7/64; A61K 38/00; A61P 33/04

(52) U.S. Cl. CPC C07K 7/64; A61P 33/04; A61K 38/00

(57) ABSTRACT

Cyclic peptides that are inhibitors of a pathogenic free-living amoeba (e.g., Balamuthia mandrillaris, Naegleria fowleri, and Acanthamoeba castellanii), compositions comprising the same, and their use for inhibiting infections or diseases caused by the pathogenic free-living amoeba.

### CROSS-REFERENCE TO RELATED APPLICATIONS

This application claims priority to U.S. provisional patent application No. 63/720,864, which was filed Nov. 15, 2024, and which is hereby incorporated by reference in its entirety.

### STATEMENT OF GOVERNMENT SUPPORT

This invention was made with government support under GM138002 and CA023168 awarded by the National Institutes of Health. The government has certain rights in the invention.

### TECHNICAL FIELD

The present disclosure relates to cyclic peptides that inhibit pathogenic free-living amoeba. In particular, the disclosure relates to cyclic peptides that are inhibitors of a specific pathogenic free-living amoeba, such as Balamuthia mandrillaris, Acanthamoeba castellanii, and Naegleria fowleri, and their use for inhibiting or treating an infection or a disease caused by them.

### BACKGROUND

This section introduces aspects that may help facilitate a better understanding of the disclosure. Accordingly, these statements are to be read in this light and are not to be construed as admissions about what is or is not prior art.

Balamuthia mandrillaris is a pathogenic and opportunistic free-living amoeba (FLA) that causes infections in humans and animals. It can initially cause cutaneous skin lesions or a presumed lung infection followed by systemic spread of infection to the central nervous system (CNS) and blood-brain barrier (BBB), causing a CNS disease called Balamuthia amoebic encephalitis (BAE). However, most cases in the US only report CNS symptoms without skin lesions, which develop quicker than when cutaneous disease presents. Time to death from neurological complications varies from weeks to months. In the United States, 109 cases were reported from 1974 to 2016. Another hot spot for BAE is Peru, which has had 55 cases reported since 1975. With the increased awareness, knowledge of at-risk populations, and development of more specific diagnostic tools (next-generation sequencing), more cases have been identified in the past 20 years, with 28 cases reported in China, as well as cases in India, Australia, Europe, East Asia, and Africa.

For BAE, there are no FDA-approved therapies. When patients are diagnosed, they have been administered various therapeutic regimens, often using a combination of antimicrobial drugs and drugs that were used in previously successful cases (see FIG. 1). The CDC-recommended treatment consists of flucytosine, pentamidine, fluconazole, sulfadiazine, azithromycin, clarithromycin, and/or miltefosine. However, the existing therapeutics are still unsuccessful, with the mortality rate in the United States being ˜92% (Clin Infect Disease, 2019, 68, 1815-1822). Overall, this high incidence of drug failure points to the great need for effective anti-Balamuthia mandrillaris therapeutics. While a few drug screens have been performed to identify novel compounds, these screens have been primarily libraries of small molecules or specific natural products such as curcumin or resveratrol.

Cyclic peptides are a growing class of therapeutics, with 18 approved by the FDA in the past two decades (RSC Chem Biol, 2022m 3, 18-31). Cyclic peptides are a promising source of therapeutics because they reside in a unique space that balances the characteristics of both small molecules and biologics, known as the Goldilocks space. They can hit challenging targets such as protein-protein interactions and often have specificity similar to biologics while having bioavailabilities and cell permeabilities that are more similar to small molecules. Furthermore, cyclic peptides have increased cell permeability and proteolytic stability compared to their linear counterparts. Peptides have demonstrated activity against multiple disease states, including antifungal, antibacterial, immunosuppressants, anticancer, and more (RSC Chem Biol, 2022m 3, 18-31; J Am Chem Soc, 2019, 141, 4167-4181; Curr Opin Chem Biol 2017, 38, 24-29).

It is an object of the present disclosure to provide cyclic peptides that are inhibitors of a pathogenic free-living amoeba and their use in inhibiting or treating an infection or a disease caused by these pathogenic free-living amoeba. This and other objects and advantages, as well as inventive features, will be apparent from the detailed description provided herein.

### SUMMARY

Provided is a cyclic peptide therapeutic agent selected from:

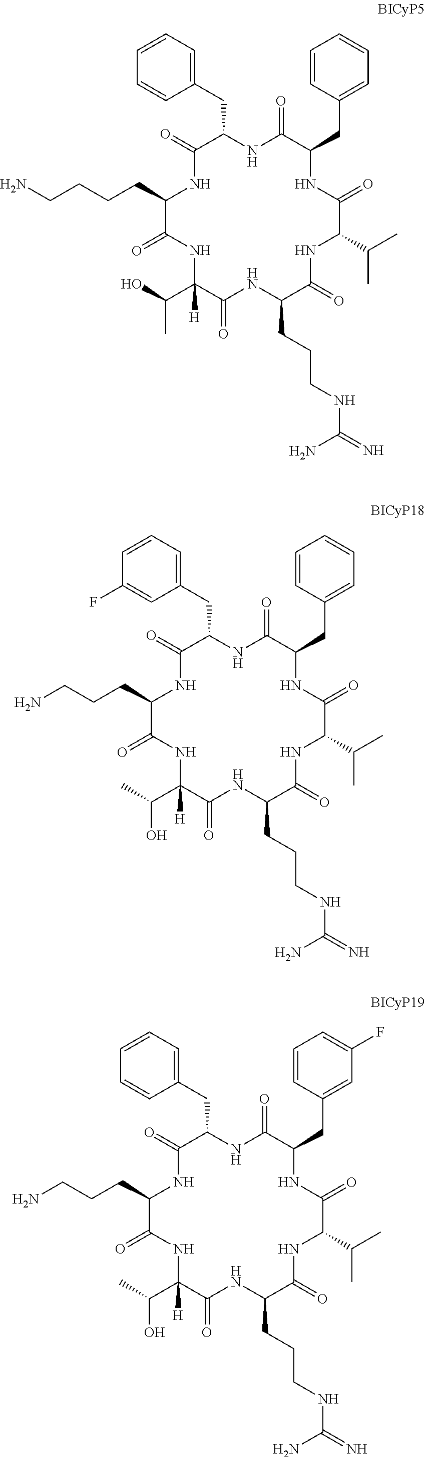 [SMILES: CC(C)[C@@H]1NC(=O)[C@@H](CCCNC(=N)N)NC(=O)[C@H]([C@@H](C)O)NC(=O)[C@@H](CCCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2cccc(F)c2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2cccc(F)c2)NC1=O]

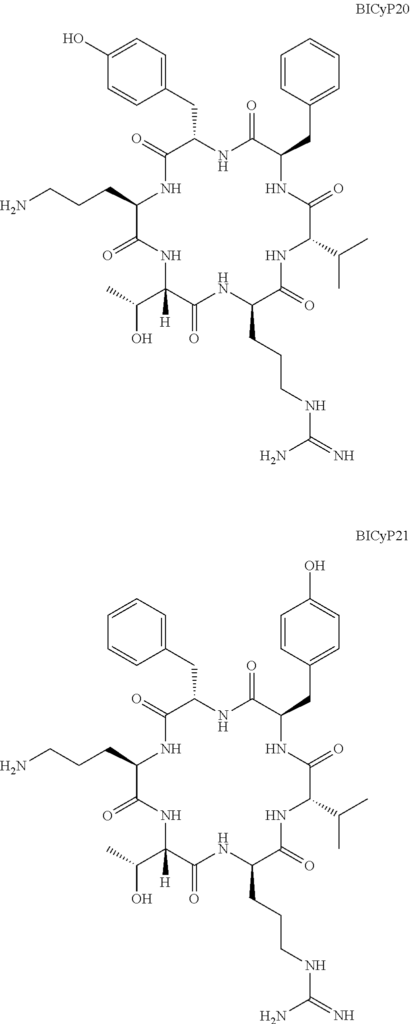 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(O)cc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(O)cc2)NC1=O]

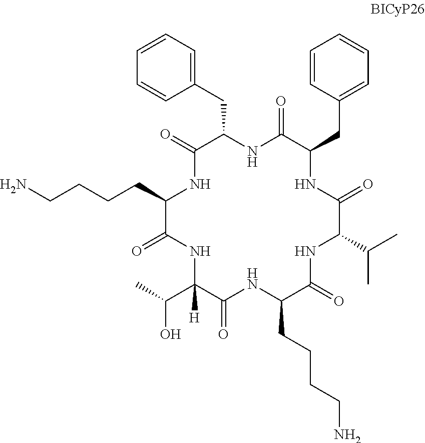 [SMILES: CC(C)[C@@H]1NC(=O)[C@@H](CCCCN)NC(=O)[C@H]([C@@H](C)O)NC(=O)[C@@H](CCCCN)NC(=O)[C@H](Cc2ccccc2)NC(=O)[C@@H](Cc2ccccc2)NC1=O]

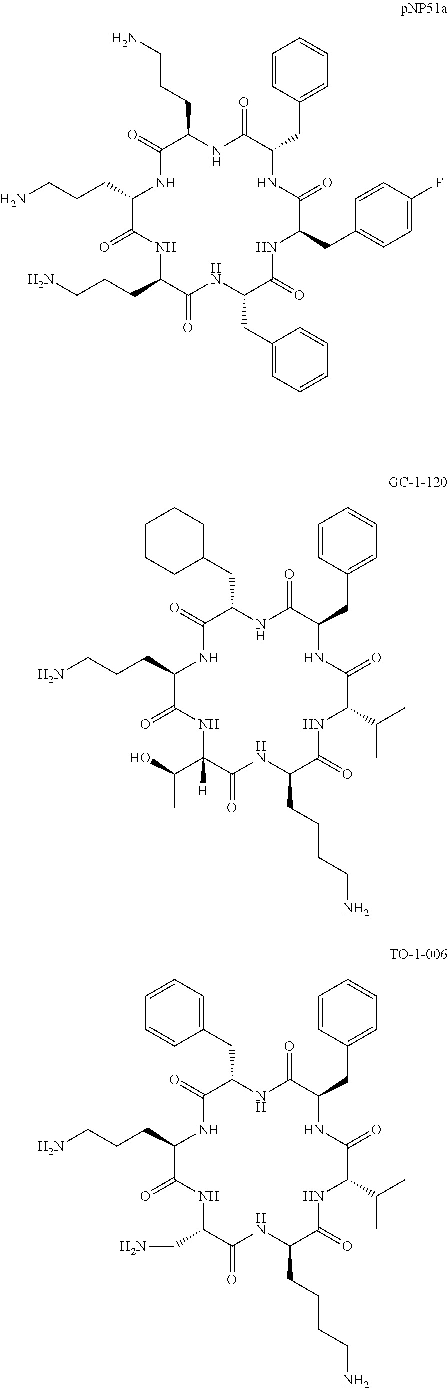 [SMILES: NCCC[C@@H]1NC(=O)[C@@H](CCCN)NC(=O)[C@H](Cc2ccccc2)NC(=O)[C@@H](Cc2ccc(F)cc2)NC(=O)[C@H](Cc2ccccc2)NC(=O)[C@@H](CCCN)NC1=O ; CC(C)[C@@H]1NC(=O)[C@@H](CCCCN)NC(=O)[C@H]([C@@H](C)O)NC(=O)[C@@H](CCCN)NC(=O)[C@H](CC2CCCCC2)NC(=O)[C@@H](Cc2ccccc2)NC1=O ; CC(C)[C@@H]1NC(=O)[C@@H](CCCCN)NC(=O)[C@H](CN)NC(=O)[C@@H](CCCN)NC(=O)[C@H](Cc2ccccc2)NC(=O)[C@@H](Cc2ccccc2)NC1=O]

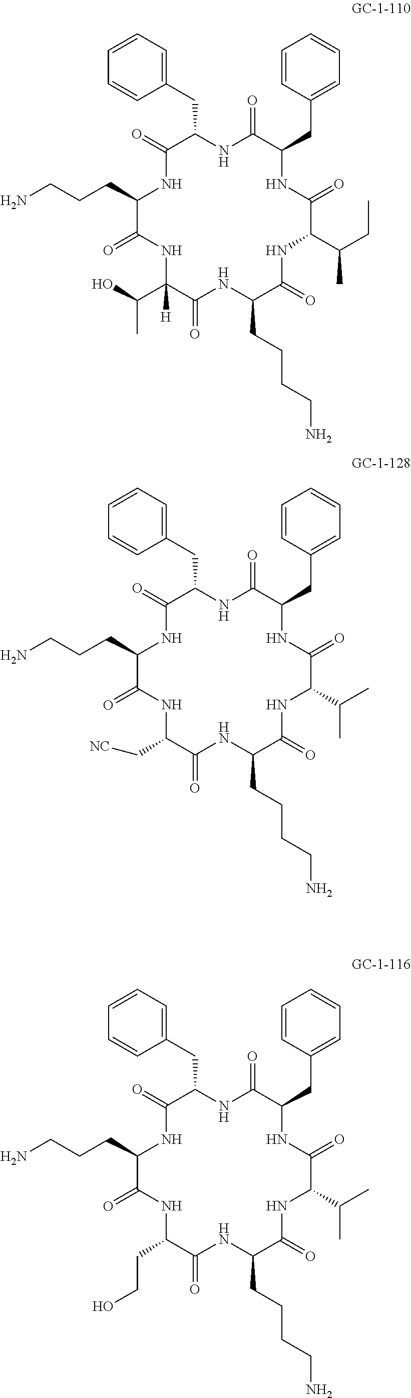 [SMILES: CC[C@@H](C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H](CC#N)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H](CCO)NC(=O)[C@@H](CCCN)NC(=O)[C@H](Cc2ccccc2)NC(=O)[C@@H](Cc2ccccc2)NC1=O]

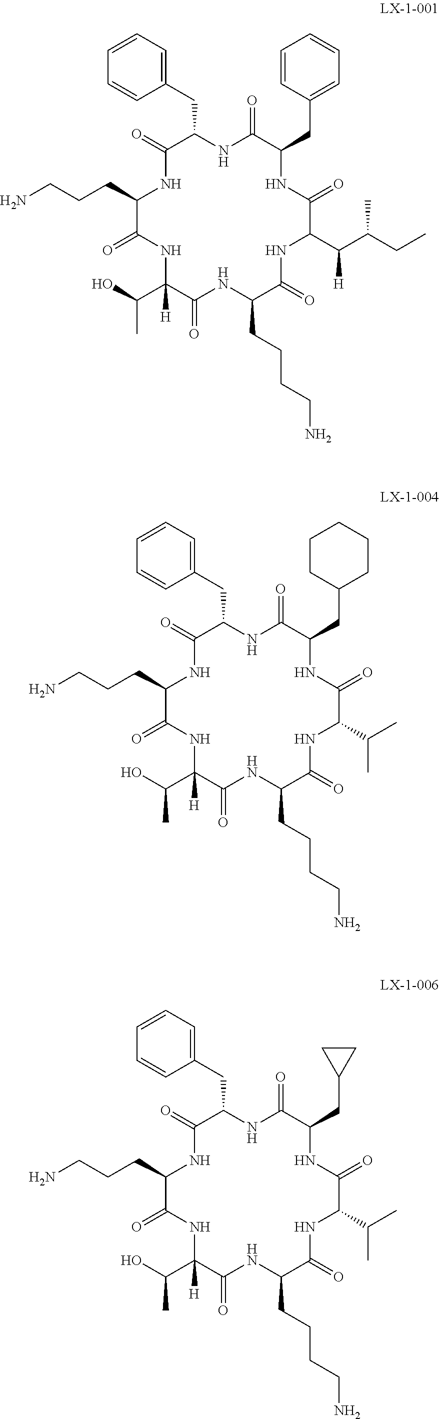

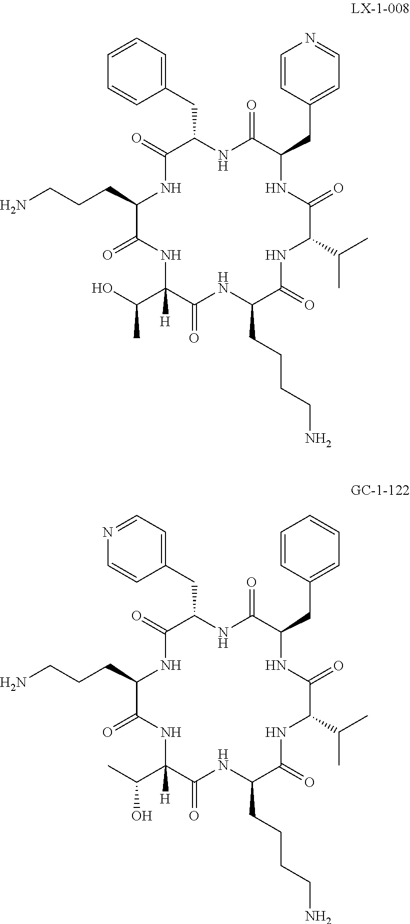 [SMILES: CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccncc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccncc2)NC(=O)[C@@H](Cc2ccccc2)NC1=O]

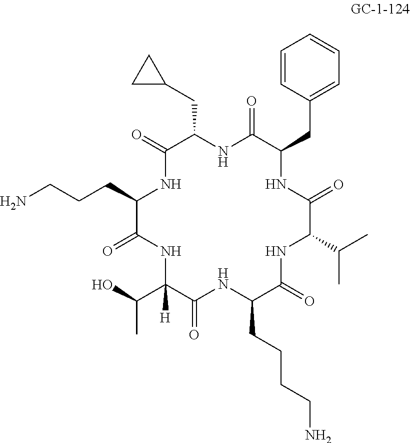 [SMILES: CC(C)[C@@H]1NC(=O)[C@@H](CCCCN)NC(=O)[C@H]([C@@H](C)O)NC(=O)[C@@H](CCCN)NC(=O)[C@H](CC2CC2)NC(=O)[C@@H](Cc2ccccc2)NC1=O]

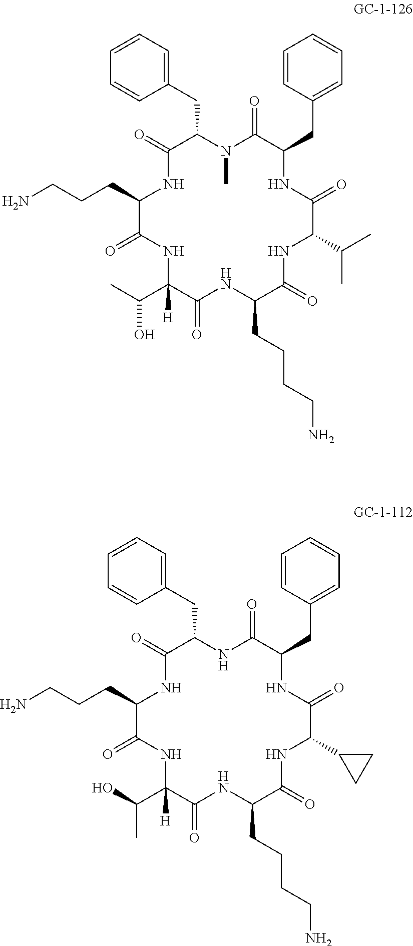

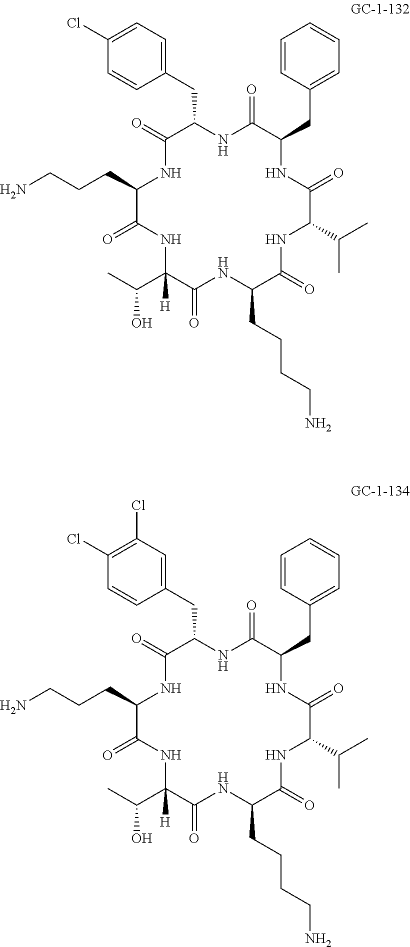 [SMILES: CC(C)[C@@H]1NC(=O)[C@@H](CCCCN)NC(=O)[C@H]([C@@H](C)O)NC(=O)[C@@H](CCCN)NC(=O)[C@H](Cc2ccc(Cl)cc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccc(Cl)c(Cl)c2)NC(=O)[C@@H](Cc2ccccc2)NC1=O]

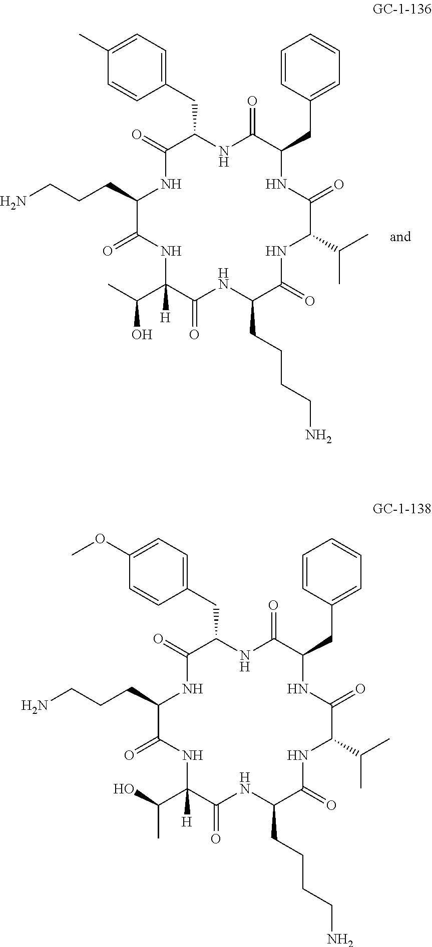 [SMILES: Cc1ccc(C[C@@H]2NC(=O)[C@@H](Cc3ccccc3)NC(=O)[C@H](C(C)C)NC(=O)[C@@H](CCCCN)NC(=O)[C@H]([C@H](C)O)NC(=O)[C@@H](CCCN)NC2=O)cc1 ; COc1ccc(C[C@@H]2NC(=O)[C@@H](Cc3ccccc3)NC(=O)[C@H](C(C)C)NC(=O)[C@@H](CCCCN)NC(=O)[C@H]([C@@H](C)O)NC(=O)[C@@H](CCCN)NC2=O)cc1]

or a variant thereof.

Provided is a pharmaceutical composition comprising (a) a cyclic peptide therapeutic agent as described above and (b) a pharmaceutically acceptable carrier, diluent, or excipient. In some embodiments, the pharmaceutical composition can further comprise one or more additional therapeutic agents, one or more of which can be other than the above-described cyclic peptide therapeutic agent.

Further provided is a method of inhibiting an infection or a disease caused by a pathogenic free-living amoeba in a patient in need thereof, which method comprises administering an inhibitory effective amount of a cyclic peptide therapeutic agent selected from

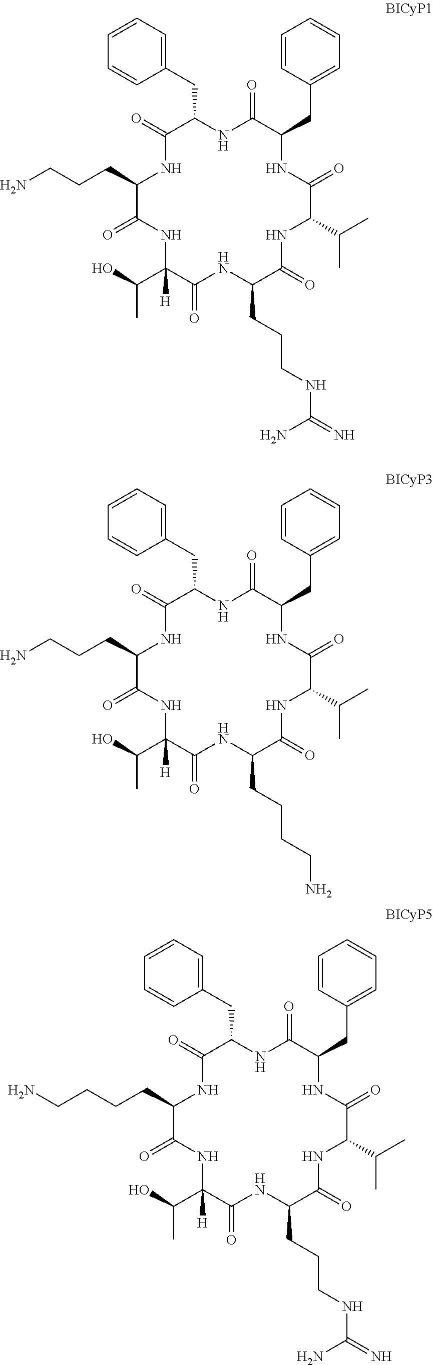 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCCN)NC(=O)[C@H](Cc2ccccc2)NC(=O)[C@@H](Cc2ccccc2)NC1=O]

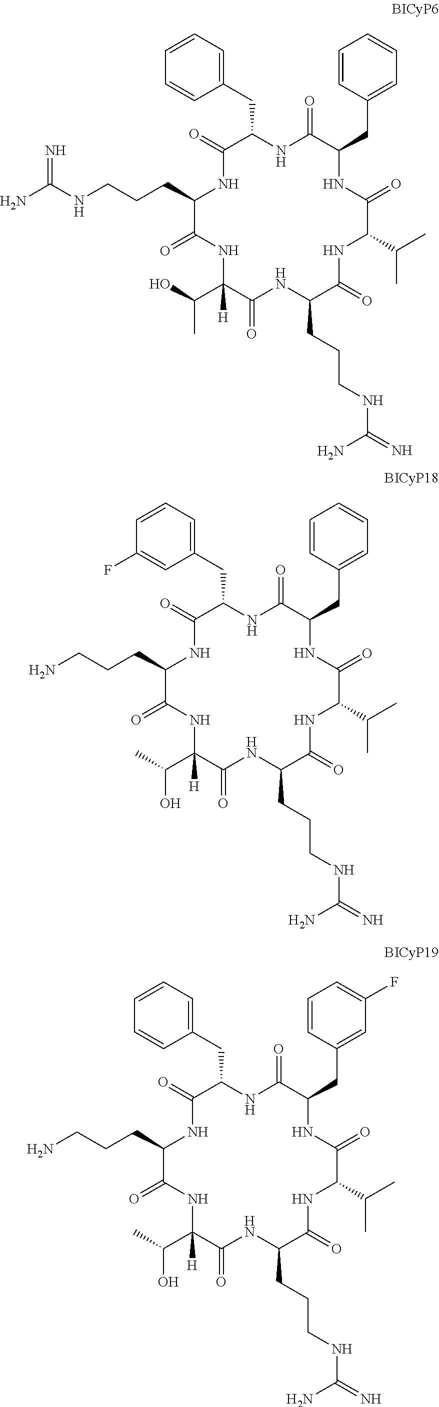 [SMILES: CC(C)[C@@H]1NC(=O)[C@@H](CCCNC(=N)N)NC(=O)[C@H]([C@@H](C)O)NC(=O)[C@@H](CCCNC(=N)N)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2cccc(F)c2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2cccc(F)c2)NC1=O]

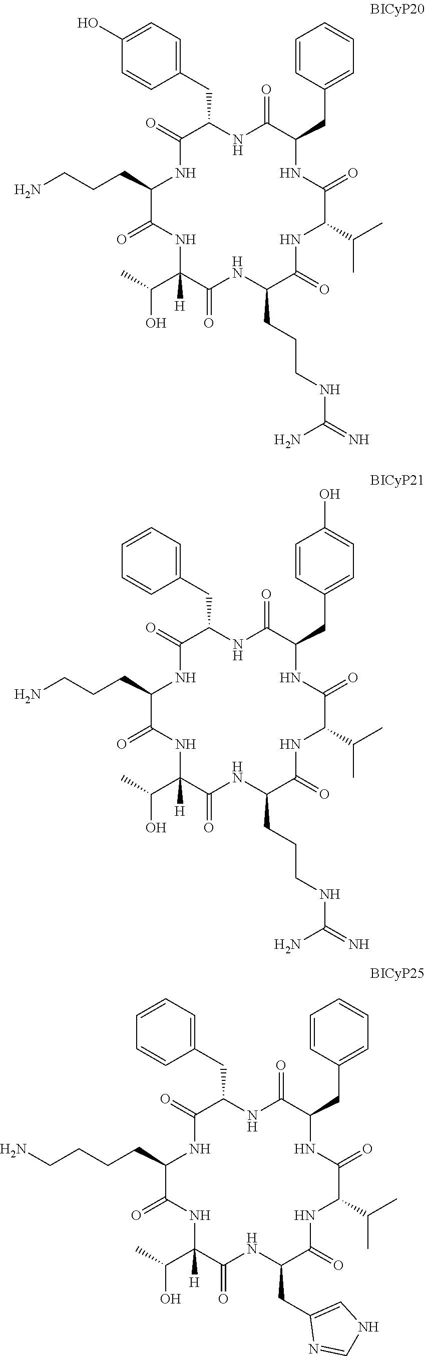 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(O)cc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(O)cc2)NC1=O ; CC(C)[C@@H]1NC(=O)[C@@H](Cc2c[nH]cn2)NC(=O)[C@H]([C@@H](C)O)NC(=O)[C@@H](CCCCN)NC(=O)[C@H](Cc2ccccc2)NC(=O)[C@@H](Cc2ccccc2)NC1=O]

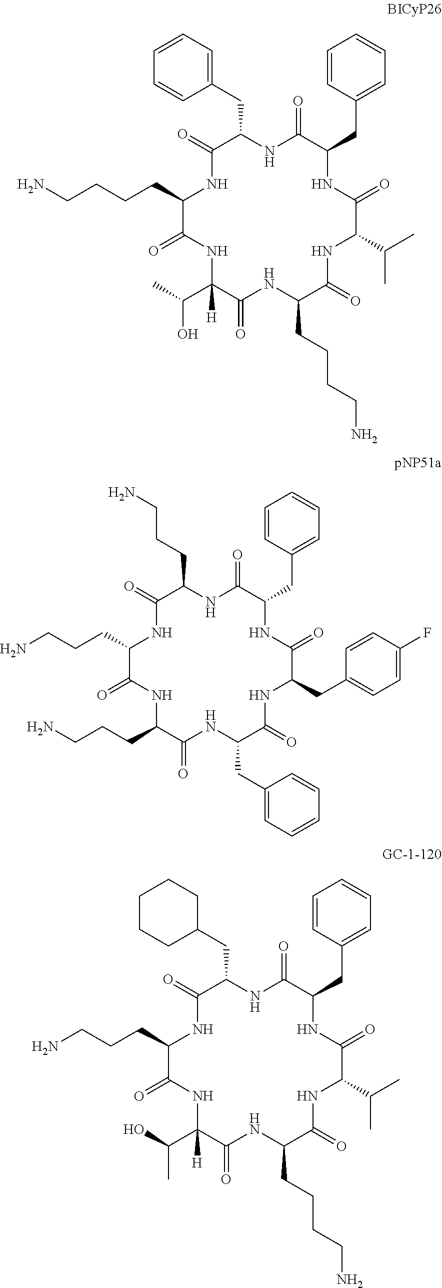 [SMILES: CC(C)[C@@H]1NC(=O)[C@@H](CCCCN)NC(=O)[C@H]([C@@H](C)O)NC(=O)[C@@H](CCCCN)NC(=O)[C@H](Cc2ccccc2)NC(=O)[C@@H](Cc2ccccc2)NC1=O ; NCCC[C@@H]1NC(=O)[C@@H](CCCN)NC(=O)[C@H](Cc2ccccc2)NC(=O)[C@@H](Cc2ccc(F)cc2)NC(=O)[C@H](Cc2ccccc2)NC(=O)[C@@H](CCCN)NC1=O ; CC(C)[C@@H]1NC(=O)[C@@H](CCCCN)NC(=O)[C@H]([C@@H](C)O)NC(=O)[C@@H](CCCN)NC(=O)[C@H](CC2CCCCC2)NC(=O)[C@@H](Cc2ccccc2)NC1=O]

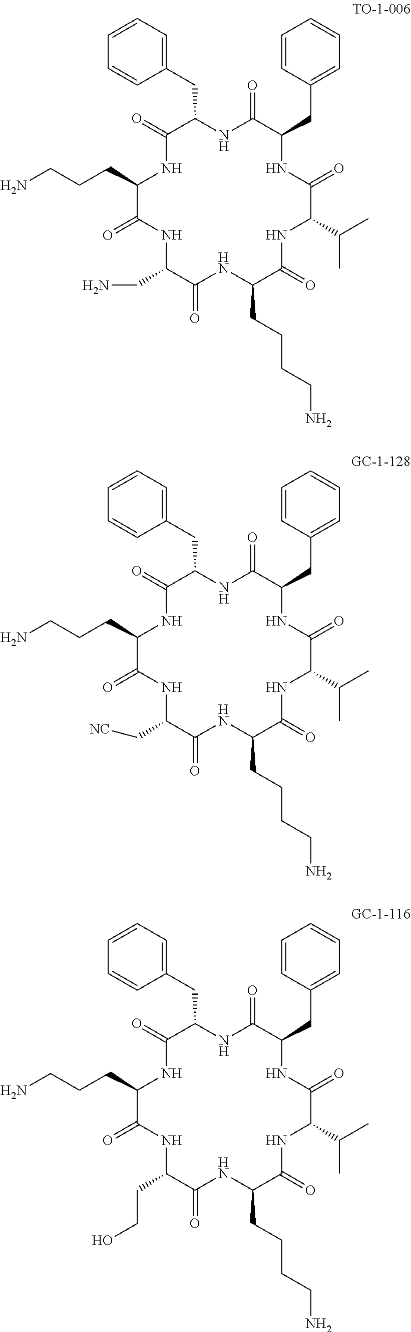 [SMILES: CC(C)[C@@H]1NC(=O)[C@@H](CCCCN)NC(=O)[C@H](CN)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H](CC#N)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H](CCO)NC(=O)[C@@H](CCCN)NC(=O)[C@H](Cc2ccccc2)NC(=O)[C@@H](Cc2ccccc2)NC1=O]

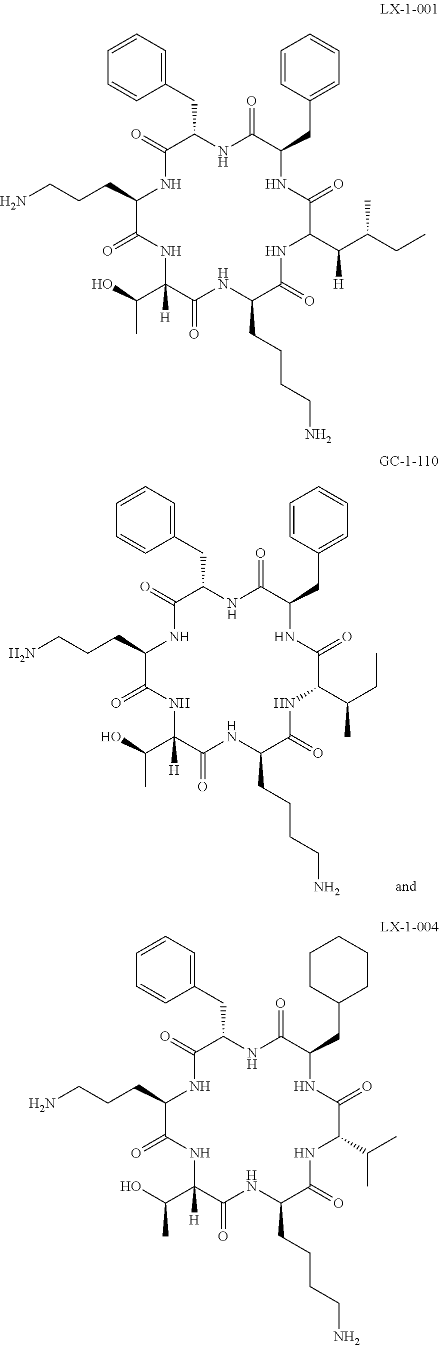

or a variant thereof, or a pharmaceutical composition comprising the cyclic peptide therapeutic agent and a pharmaceutically acceptable carrier, diluent, or excipient, whereupon the infection or disease caused by the pathogenic free-living amoeba in the patient is inhibited.

The infection can be an infection of the central nervous system. In some embodiments, the patient has Balamuthia amoebic encephalitis (BAE). In some embodiments, the infection can be cutaneous or systemic. In some embodiments, the pathogenic free-living amoeba is Balamuthia mandrillaris. In some embodiments, the pathogenic free-living amoeba is Naegleria fowleri. In some embodiments, the pathogenic free-living amoeba is Acanthamoeba castellanii.

### BRIEF DESCRIPTION OF THE DRAWINGS

The present disclosure will be more readily understood from the detailed description of embodiments presented below considered in conjunction with the attached drawings of which:

FIG. 1 shows the structures and IC₅₀s of the Centers for Disease Control and Prevention (CDC) recommended treatments for Balamuthia mandrillaris infections.

FIG. 2A shows the Tanimoto similarity tree of the peptide library reported in ACS Chem Biol, 2021, 16, 2604-2611. All circles represent compounds tested. Black circle depicts hits against Balamuthia mandrillaris, grey circle depicts peptides active against Acanthamoeba castellanii, and triangle depicts cyclic peptides that inhibit Naegleria fowleri. All hits have their designated compound (pNP) number next to their respective circle.

FIG. 2B shows the structures of the top eight initial hits against Balamuthia mandrillaris. Of the top eight hits, six are derivatives of compound BICyP1.

FIG. 3A shows the structure-activity relationship of compound BICyP1.

FIG. 3B shows the dose-response curve for the most active variant BICyP3 and pentamidine in the Balamuthia mandrillaris activity assay.

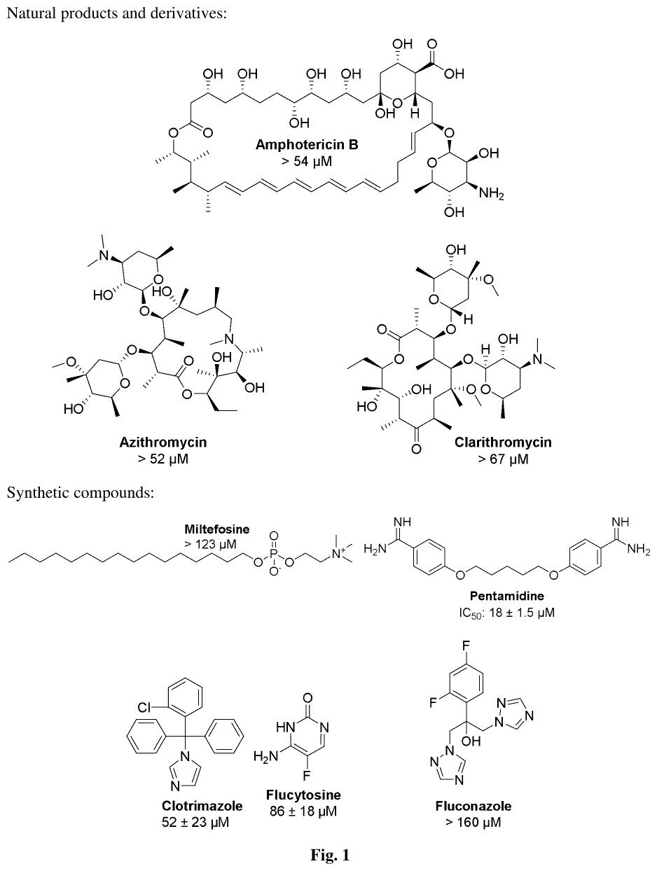

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The present disclosure is predicated, at least in part, on the discovery that the peptides can have activity against free-living amoebas (FLAs), for example, Naegleria fowleri, Acanthamoeba globifora, and Acanthamoeba castellanii (Journal of Trop Med Public Health, 2014, 45, 537-46; ACS Omega 2022, 7, 28797-28805; Int J Parasitol Drugs Drug Resist, 2021, 17, 67-79; Microorganisms 10, 2022; Acta Parasitol, 2022, 67, 511-517) but none have demonstrated activity against Balamuthia mandrillaris. Specifically, a tyrocidine-derived linear peptide exhibited IC₅₀ of 112 μg/mL (88 μM) against Acanthamoeba castellanii and 82 μg/mL (64 μM) against Naegleria fowleri at 24 hours. Nisin, a ribosomally synthesized post-translationally modified peptide produced by Lactococcus lactis, appears to affect the growth of Acanthamoeba, resulting in smaller and rounder trophozoites. However, the trophozoites proliferated again after 72 hours. Overall, the IC₅₀ of nisin was quite poor (4493.2 ILU/mL, 4.5 mg/mL at 24 h), suggesting that it is unlikely to be an effective treatment for Acanthamoeba.

In view of the above, provided is a pharmaceutical composition comprising: (a) a cyclic peptide therapeutic agent of formula (I):

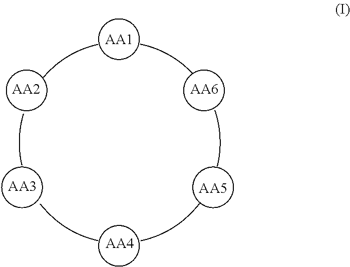

wherein:
each of AA1, AA2, AA3, AA4, AA5, and AA6 is independently an amino acid selected from phenylalanine (Phe), ornithine (Orn), threonine (Thr), arginine (Arg), valine (Val), alanine (Ala), 3-fluorophenylalanine (Phe, 3-F), lysine (Lys), histidine (His), serine (Ser), diaminobutyric acid (Dab), diaminopropionic acid (DAP), isoleucine (Ile), glycine (gly), and tryptophan (Trp), and (b) a pharmaceutically acceptable carrier, diluent, or excipient.

In some embodiments, the cyclic peptide therapeutic agent of formula (I) is BICyP1, a cyclic peptide of formula (II):

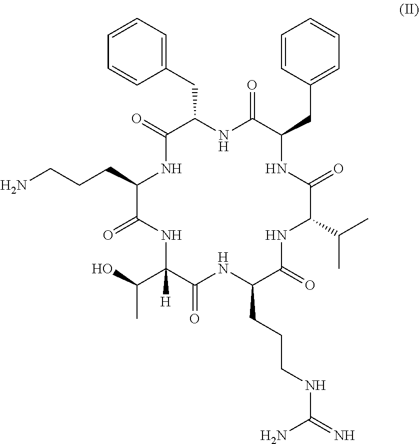

wherein, AA1 is Phe, AA2 is D-Orn, AA3 is Thr, AA4 is D-Arg, AA5 is Val, and AA6 is D-Phe. or a variant of the cyclic peptide of formula (II) selected from:

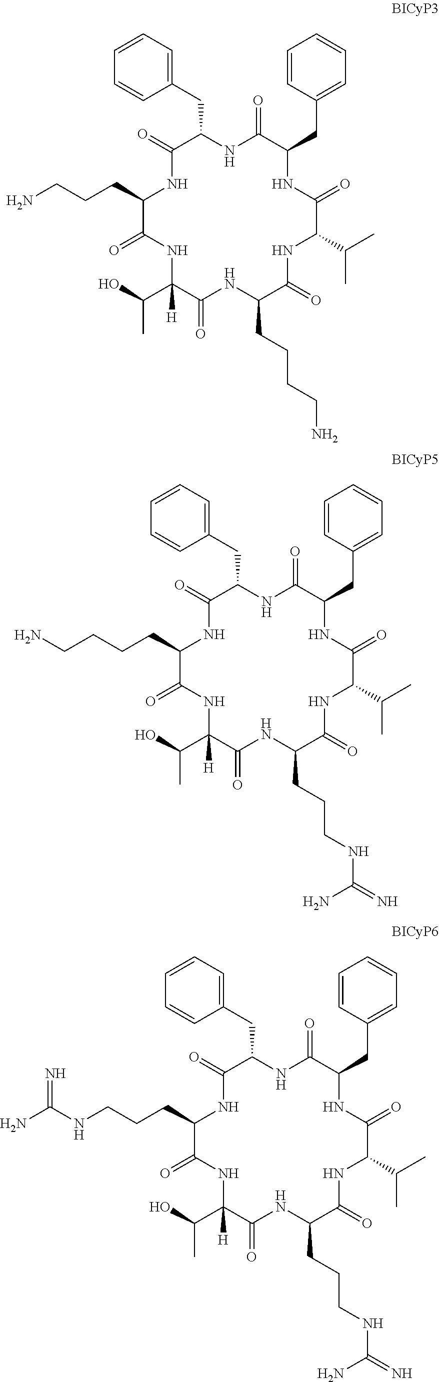 [SMILES: CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCNC(=N)N)NC(=O)[C@H](Cc2ccccc2)NC(=O)[C@@H](Cc2ccccc2)NC1=O]

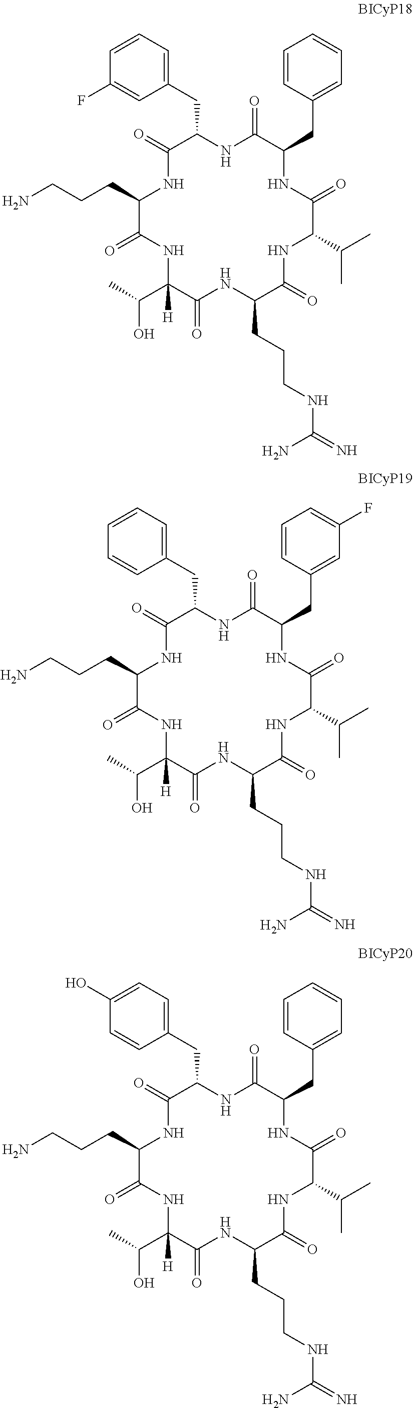 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2cccc(F)c2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2cccc(F)c2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(O)cc2)NC(=O)[C@@H](Cc2ccccc2)NC1=O]

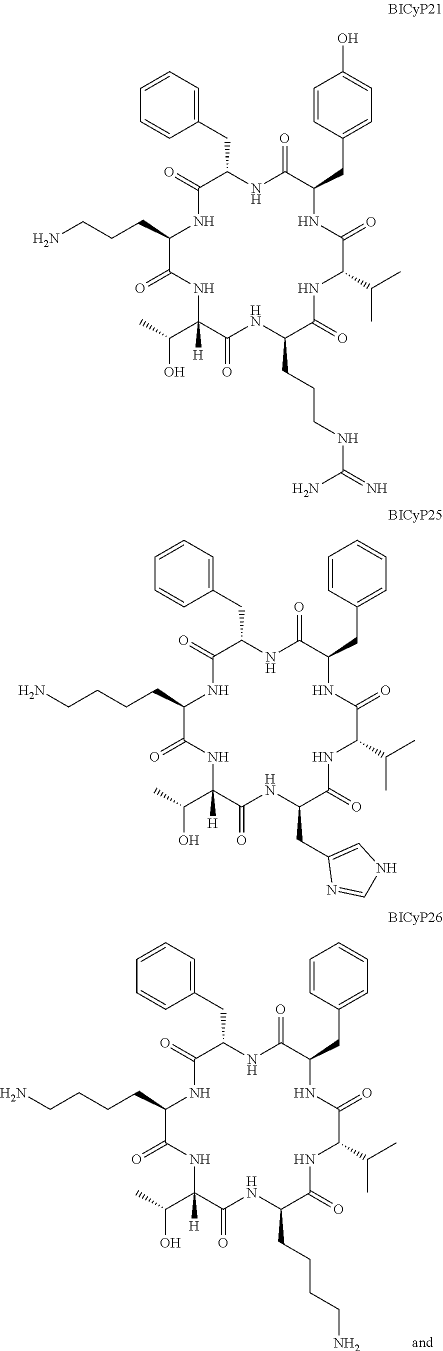 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(O)cc2)NC1=O ; CC(C)[C@@H]1NC(=O)[C@@H](Cc2c[nH]cn2)NC(=O)[C@H]([C@@H](C)O)NC(=O)[C@@H](CCCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCCN)NC(=O)[C@H](Cc2ccccc2)NC(=O)[C@@H](Cc2ccccc2)NC1=O]

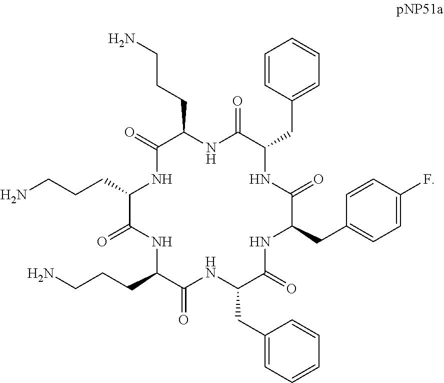 [SMILES: NCCC[C@@H]1NC(=O)[C@@H](CCCN)NC(=O)[C@H](Cc2ccccc2)NC(=O)[C@@H](Cc2ccc(F)cc2)NC(=O)[C@H](Cc2ccccc2)NC(=O)[C@@H](CCCN)NC1=O]

The BICyP1 and its variant can inhibit or treat infections or diseases caused by the pathogenic free-living amoeba. The pathogenic free-living amoeba can be a specific free-living amoeba, but not limited to Balamuthia mandrillaris (B. mandrillaris), Naegleria fowleri, and Acanthamoeba castellanii. Pathogenic free-living amoeba causes infection of the central nervous system and causes cutaneous and systemic diseases. Examples of the infections or diseases caused by these free-living amoeba include, but are not limited to, Balamuthia amoebic encephalitis (BAE), granulomatous amoebic encephalitis (GAE), primary amoebic meningoencephalitis (PAM), keratitis, lesions in skin and respiratory mucosa.

Provided is a cyclic peptide therapeutic agent selected from:

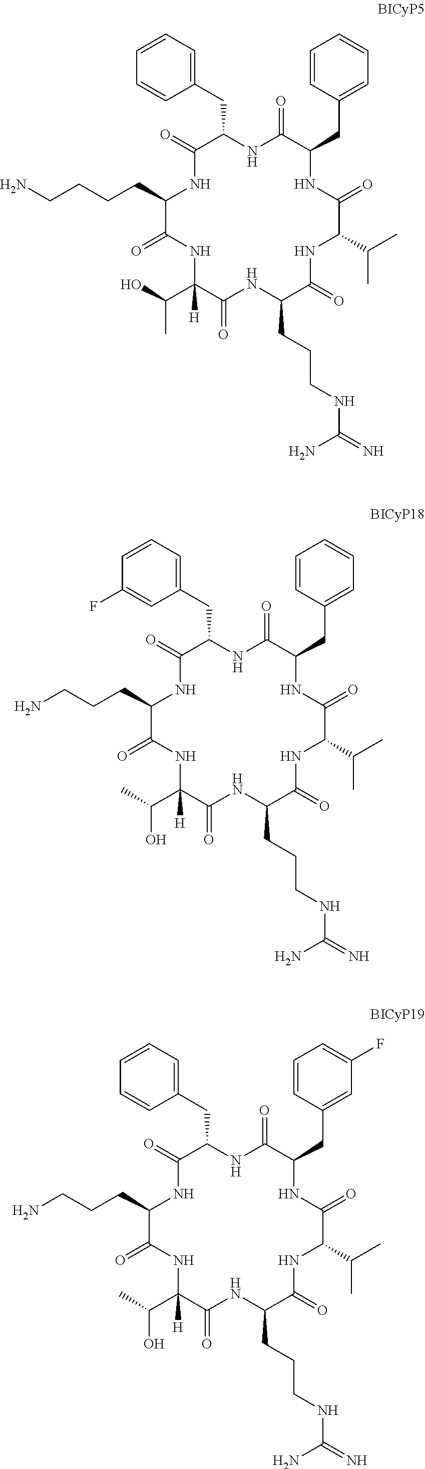 [SMILES: CC(C)[C@@H]1NC(=O)[C@@H](CCCNC(=N)N)NC(=O)[C@H]([C@@H](C)O)NC(=O)[C@@H](CCCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2cccc(F)c2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2cccc(F)c2)NC1=O]

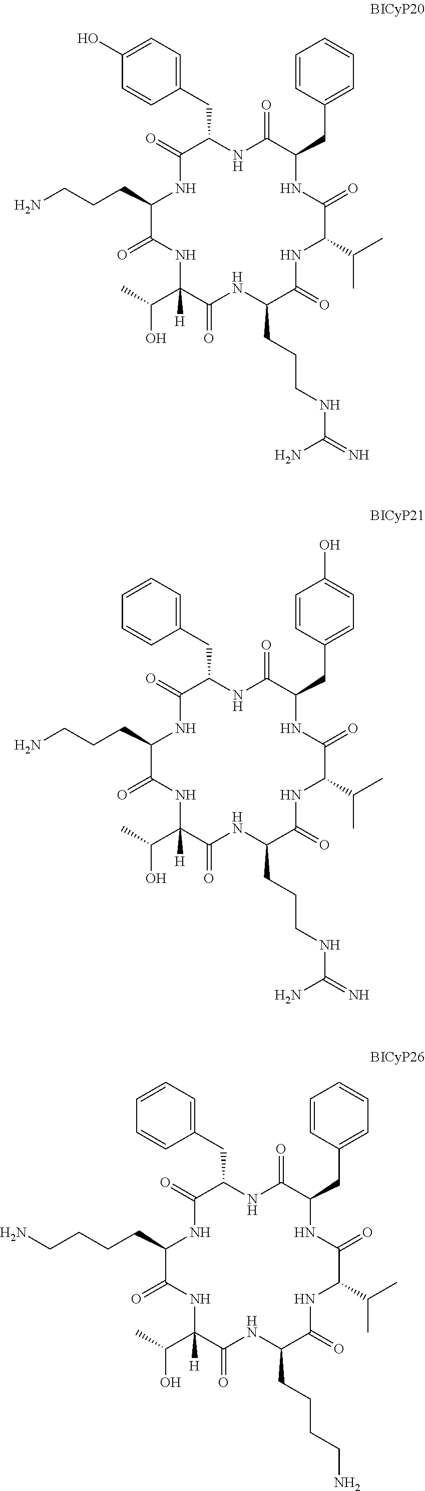 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(O)cc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(O)cc2)NC1=O ; CC(C)[C@@H]1NC(=O)[C@@H](CCCCN)NC(=O)[C@H]([C@@H](C)O)NC(=O)[C@@H](CCCCN)NC(=O)[C@H](Cc2ccccc2)NC(=O)[C@@H](Cc2ccccc2)NC1=O]

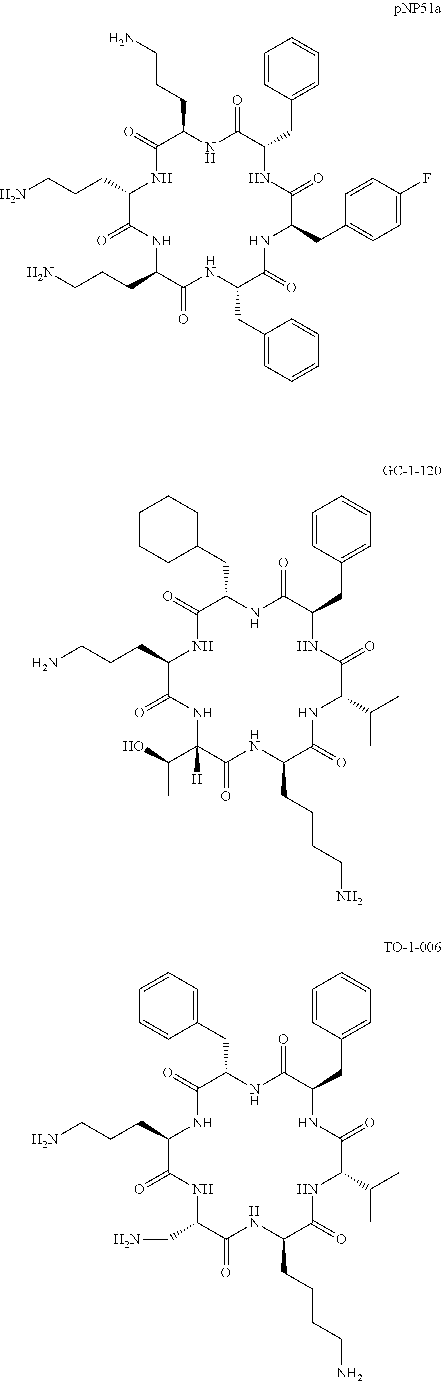 [SMILES: NCCC[C@@H]1NC(=O)[C@@H](CCCN)NC(=O)[C@H](Cc2ccccc2)NC(=O)[C@@H](Cc2ccc(F)cc2)NC(=O)[C@H](Cc2ccccc2)NC(=O)[C@@H](CCCN)NC1=O ; CC(C)[C@@H]1NC(=O)[C@@H](CCCCN)NC(=O)[C@H]([C@@H](C)O)NC(=O)[C@@H](CCCN)NC(=O)[C@H](CC2CCCCC2)NC(=O)[C@@H](Cc2ccccc2)NC1=O ; CC(C)[C@@H]1NC(=O)[C@@H](CCCCN)NC(=O)[C@H](CN)NC(=O)[C@@H](CCCN)NC(=O)[C@H](Cc2ccccc2)NC(=O)[C@@H](Cc2ccccc2)NC1=O]

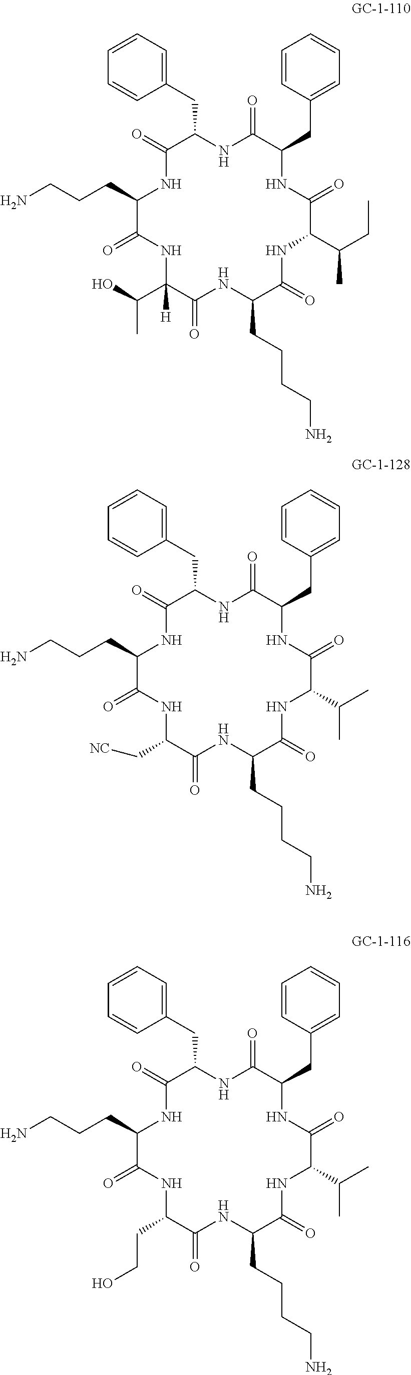 [SMILES: CC[C@@H](C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H](CC#N)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H](CCO)NC(=O)[C@@H](CCCN)NC(=O)[C@H](Cc2ccccc2)NC(=O)[C@@H](Cc2ccccc2)NC1=O]

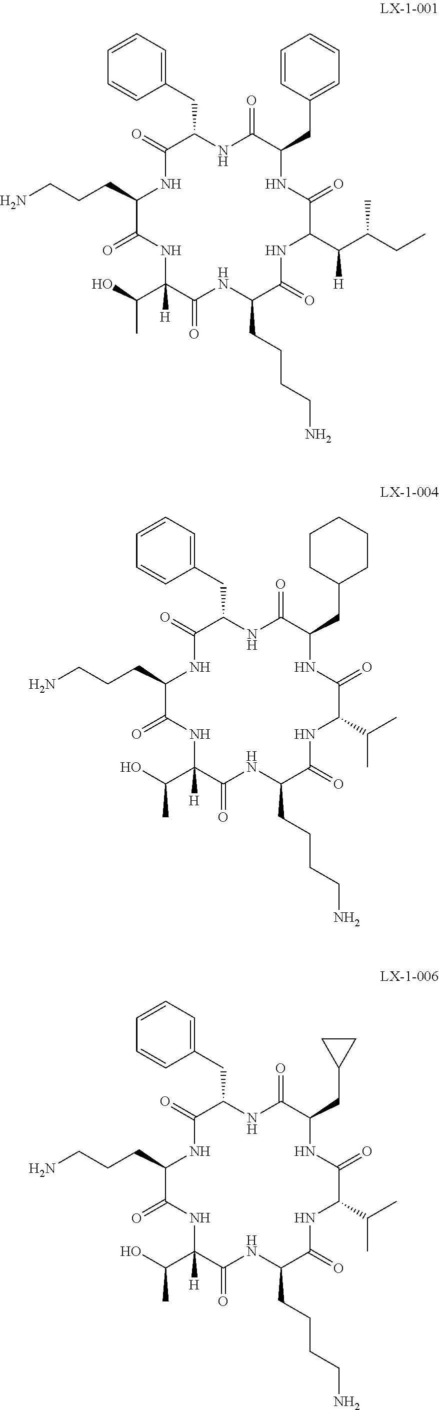

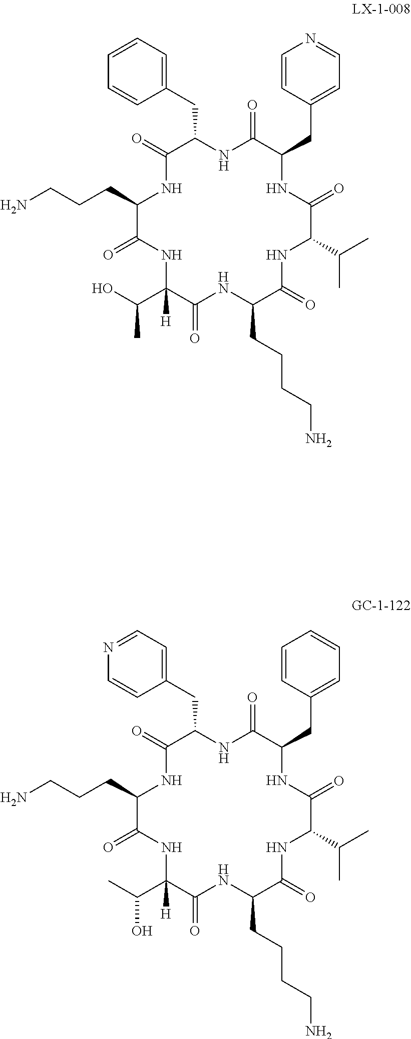 [SMILES: CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccncc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccncc2)NC(=O)[C@@H](Cc2ccccc2)NC1=O]

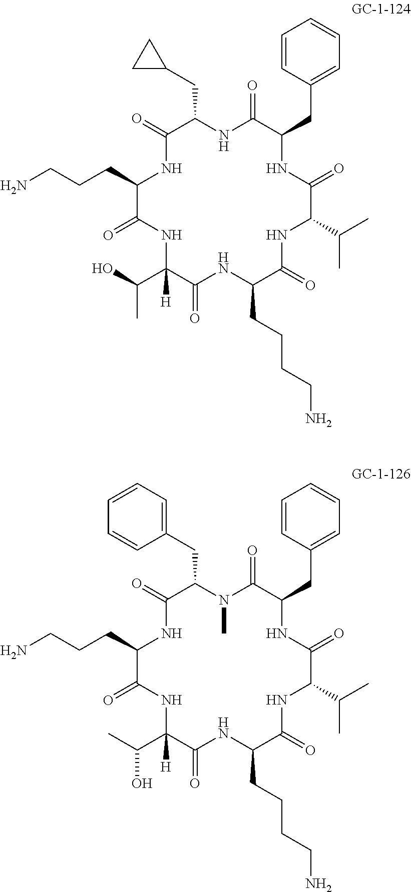

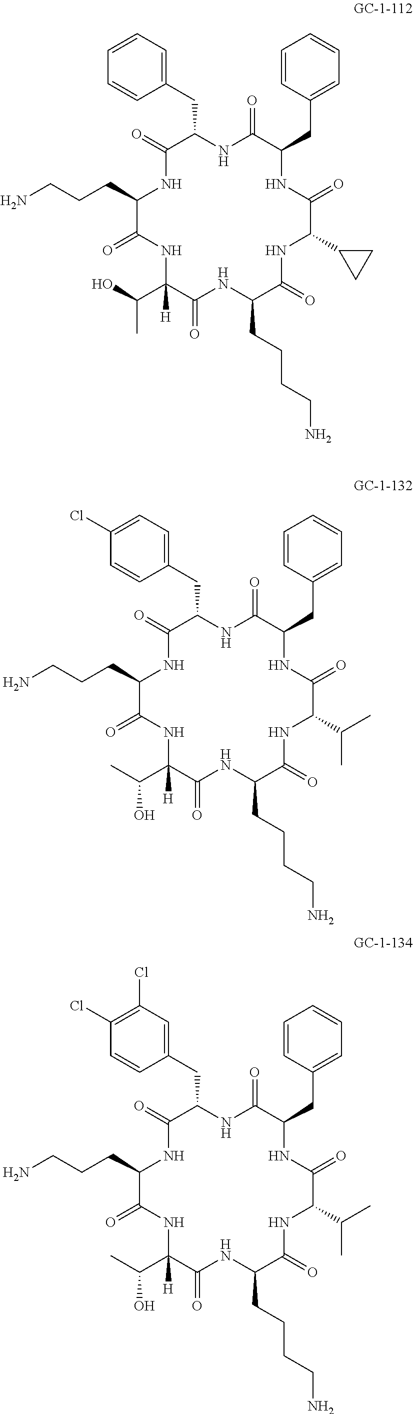 [SMILES: C[C@@H](O)[C@@H]1NC(=O)[C@@H](CCCN)NC(=O)[C@H](Cc2ccccc2)NC(=O)[C@@H](Cc2ccccc2)NC(=O)[C@H](C2CC2)NC(=O)[C@@H](CCCCN)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccc(Cl)cc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccc(Cl)c(Cl)c2)NC(=O)[C@@H](Cc2ccccc2)NC1=O]

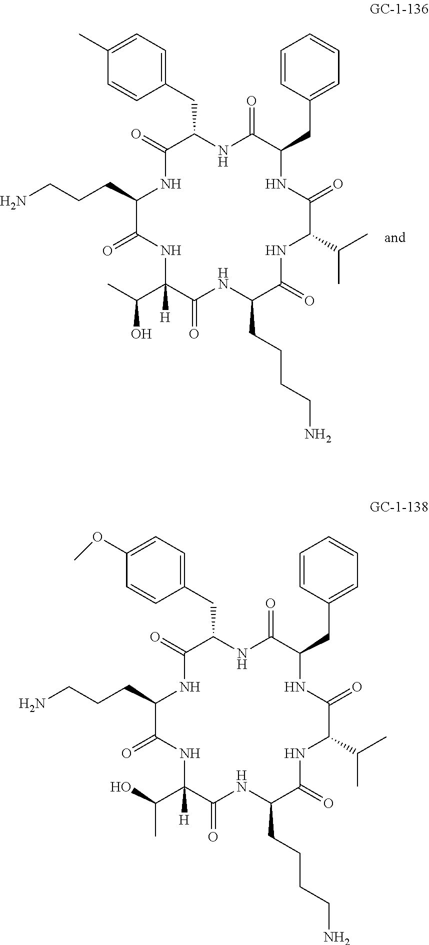 [SMILES: Cc1ccc(C[C@@H]2NC(=O)[C@@H](Cc3ccccc3)NC(=O)[C@H](C(C)C)NC(=O)[C@@H](CCCCN)NC(=O)[C@H]([C@H](C)O)NC(=O)[C@@H](CCCN)NC2=O)cc1 ; COc1ccc(C[C@@H]2NC(=O)[C@@H](Cc3ccccc3)NC(=O)[C@H](C(C)C)NC(=O)[C@@H](CCCCN)NC(=O)[C@H]([C@@H](C)O)NC(=O)[C@@H](CCCN)NC2=O)cc1]

or a variant thereof.

Provided is a pharmaceutical composition comprising (a) a cyclic peptide therapeutic agent selected from BICyP5, BICyP18, BICyP19, BICyP20, BICyP21, BICyP26, pNP51a, GC-1-110, GC-1-112, GC-1-116, GC-1-120, GC-1-122, GC-1-124, GC-1-126, GC-1-128, TO-1-006, LX-1-001, LX-1-004, LX-1-006, LX-1-008, GC-1-132, GC-1-134, GC-1-136, and GC-1-138 and (b) a pharmaceutically acceptable carrier, diluent, or excipient.

In some embodiments, the pharmaceutical compositions can further comprise one or more additional therapeutic agents and one or more pharmaceutically acceptable carriers, diluents, or excipients. In some embodiments, one or more additional therapeutic agents can be other than a cyclic peptide therapeutic agent. The carriers, excipients, or diluents can vary based on the particular route of administration (see, e.g., Remington's The Science and Practice of Pharmacy, 23^{rd} ed. (2020)).

Provided is a method for inhibiting an infection or a disease caused by a pathogenic free-living amoeba in a patient in need thereof, which method comprises administering to the patient an inhibitory effective amount of a cyclic peptide therapeutic agent selected from

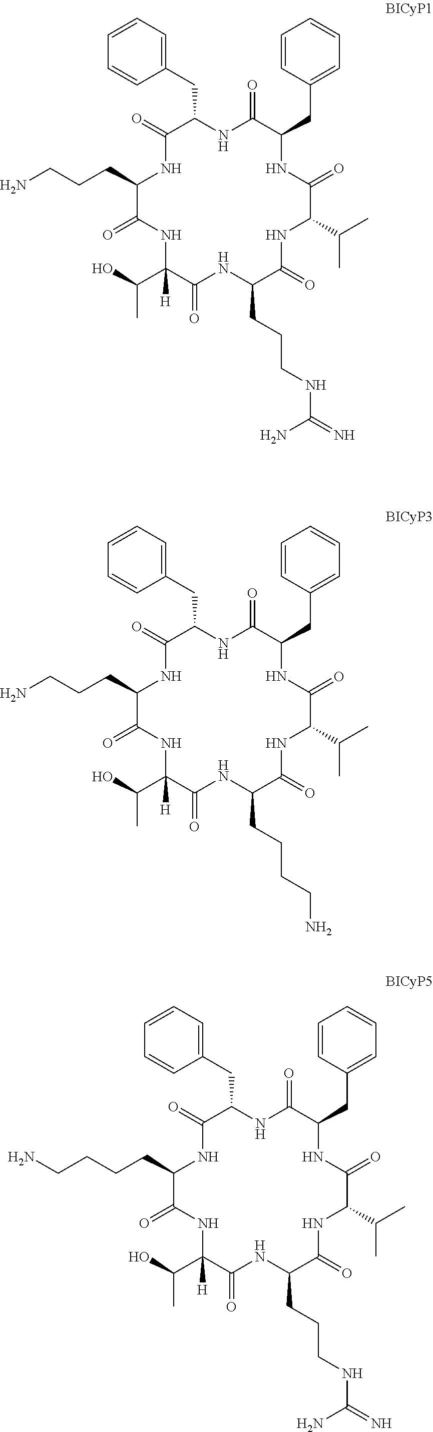 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCCN)NC(=O)[C@H](Cc2ccccc2)NC(=O)[C@@H](Cc2ccccc2)NC1=O]

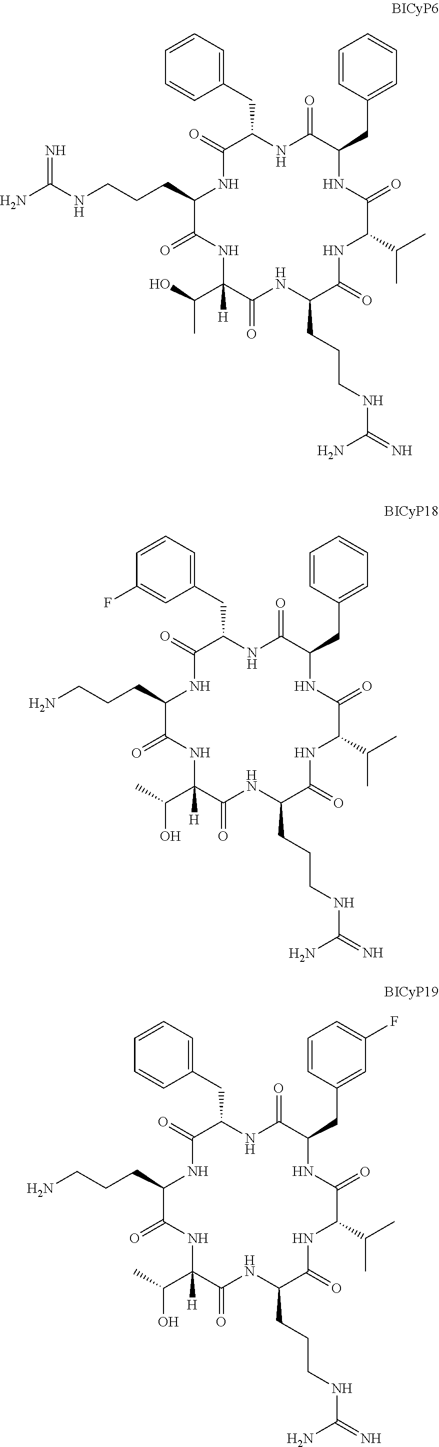 [SMILES: CC(C)[C@@H]1NC(=O)[C@@H](CCCNC(=N)N)NC(=O)[C@H]([C@@H](C)O)NC(=O)[C@@H](CCCNC(=N)N)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2cccc(F)c2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2cccc(F)c2)NC1=O]

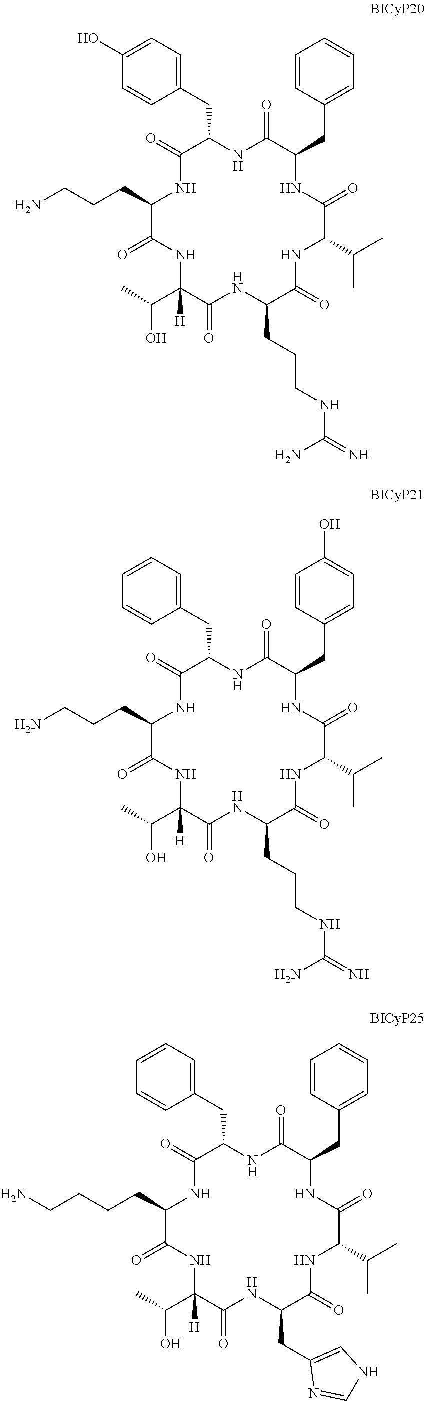 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(O)cc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(O)cc2)NC1=O ; CC(C)[C@@H]1NC(=O)[C@@H](Cc2c[nH]cn2)NC(=O)[C@H]([C@@H](C)O)NC(=O)[C@@H](CCCCN)NC(=O)[C@H](Cc2ccccc2)NC(=O)[C@@H](Cc2ccccc2)NC1=O]

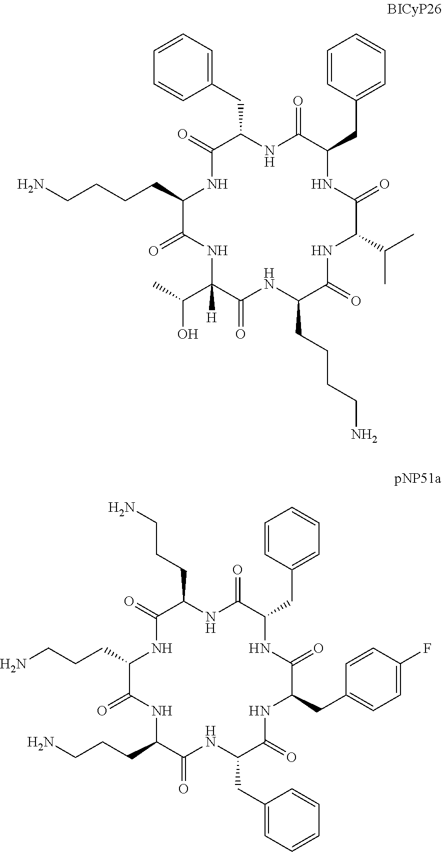 [SMILES: CC(C)[C@@H]1NC(=O)[C@@H](CCCCN)NC(=O)[C@H]([C@@H](C)O)NC(=O)[C@@H](CCCCN)NC(=O)[C@H](Cc2ccccc2)NC(=O)[C@@H](Cc2ccccc2)NC1=O ; NCCC[C@@H]1NC(=O)[C@@H](CCCN)NC(=O)[C@H](Cc2ccccc2)NC(=O)[C@@H](Cc2ccc(F)cc2)NC(=O)[C@H](Cc2ccccc2)NC(=O)[C@@H](CCCN)NC1=O]

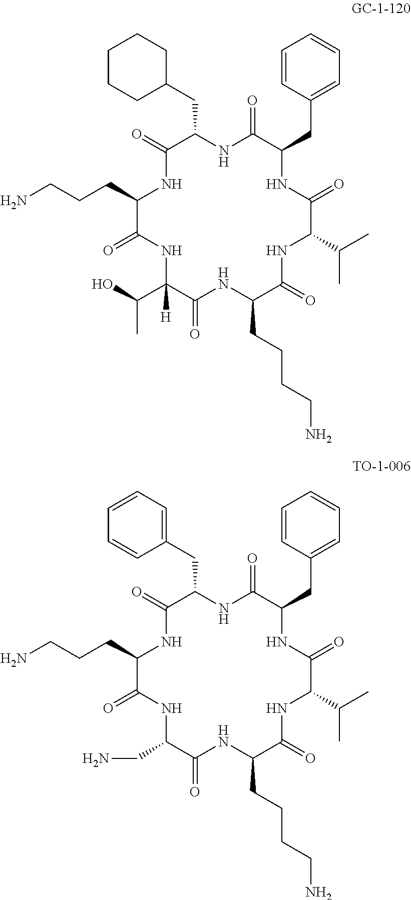 [SMILES: CC(C)[C@@H]1NC(=O)[C@@H](CCCCN)NC(=O)[C@H]([C@@H](C)O)NC(=O)[C@@H](CCCN)NC(=O)[C@H](CC2CCCCC2)NC(=O)[C@@H](Cc2ccccc2)NC1=O ; CC(C)[C@@H]1NC(=O)[C@@H](CCCCN)NC(=O)[C@H](CN)NC(=O)[C@@H](CCCN)NC(=O)[C@H](Cc2ccccc2)NC(=O)[C@@H](Cc2ccccc2)NC1=O]

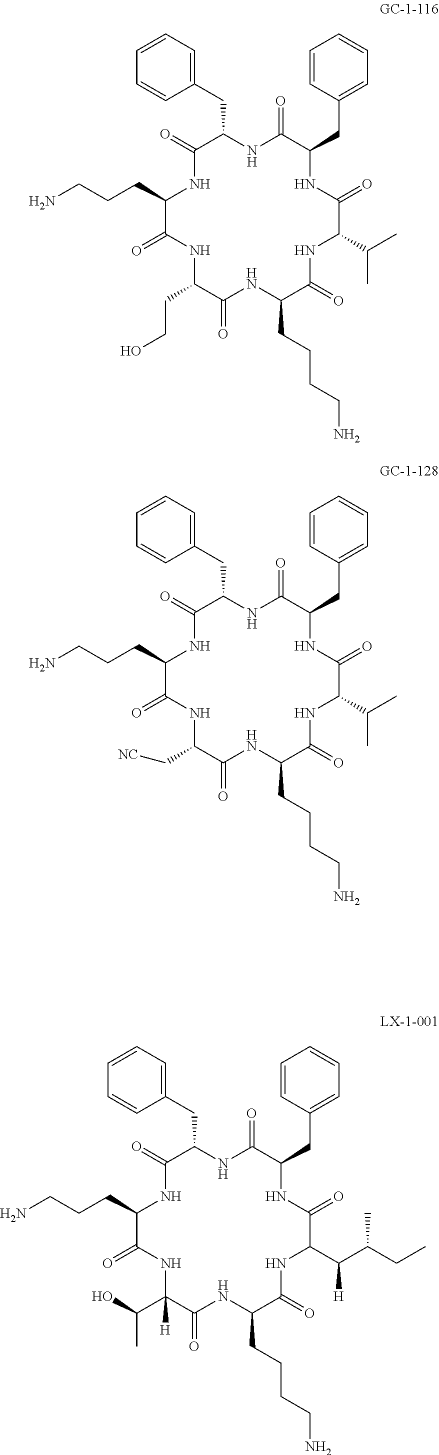

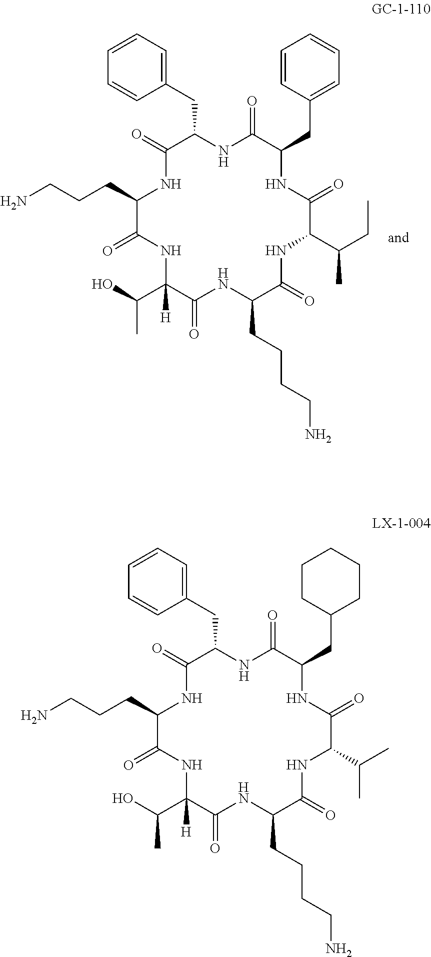 [SMILES: CC[C@@H](C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](CC2CCCCC2)NC1=O]

or a variant thereof or a pharmaceutical composition comprising the same, whereupon the infection or disease caused by the pathogenic free-living amoeba in the patient is inhibited.

In some embodiments, the patient has an infection of the central nervous system. In some embodiments, the patient has cutaneous or systemic disease. In some embodiments, the patient has an infection of the central nervous system, such as Balamuthia amoebic encephalitis (BAE). In some embodiments, the pathogenic free-living amoeba is Balamuthia mandrillaris. In some embodiments, the pathogenic free-living amoeba is Naegleria fowleri. In some embodiments, the pathogenic free-living amoeba is Acanthamoeba castellanii.

The other variants of BICyP1 of formula (II) screened for potency and toxicity by performing alanine scan are:

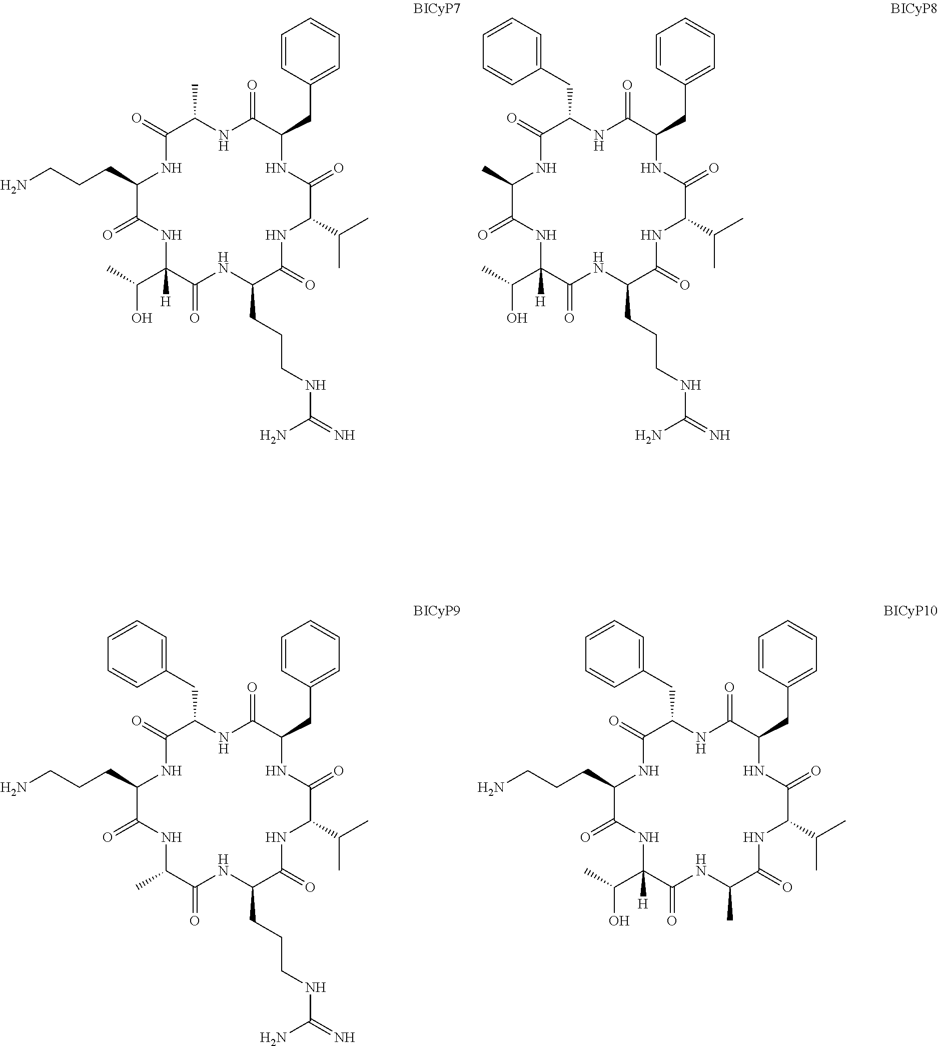 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](C)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](C)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H](C)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](C)NC(=O)[C@H]([C@@H](C)O)NC(=O)[C@@H](CCCN)NC(=O)[C@H](Cc2ccccc2)NC(=O)[C@@H](Cc2ccccc2)NC1=O]

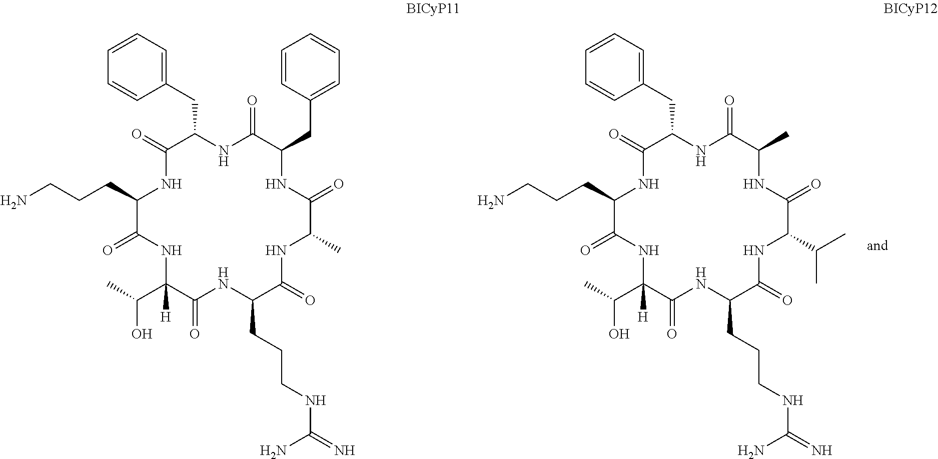 [SMILES: C[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](C)NC1=O]

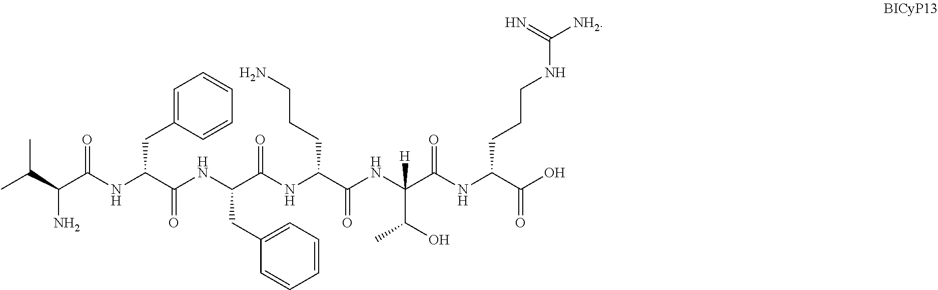 [SMILES: CC(C)[C@H](N)C(=O)N[C@H](Cc1ccccc1)C(=O)N[C@@H](Cc1ccccc1)C(=O)N[C@H](CCCN)C(=O)N[C@H](C(=O)N[C@H](CCCNC(=N)N)C(=O)O)[C@@H](C)O]

The other variants of BICyP1 of formula (II) are:
BICyP2, wherein AA4 in formula (II) is D-Orn; BICyP4, wherein AA4 in formula (II) is D-Dab; BICyP14, wherein AA1 in formula (II) is Phe (4F); BICyP15, wherein AA6 in formula (II) is D-Phe (4F); BICyP16, wherein AA1 in formula (II) is Phe (3, 4F); BICyP17, wherein AA6 in formula (II) is D-Phe (3, 4F); BICyP22, wherein AA2 in formula (II) is D-Dap; BICyP23, wherein AA2 in formula (II) is D-Dab; BICyP24, wherein AA4 in formula (II) is D-His; or BICyP27, wherein AA2 in formula (II) is D-Lys and AA4 is D-Trp; or BICyP28, wherein AA5 in formula (II) is Pra.

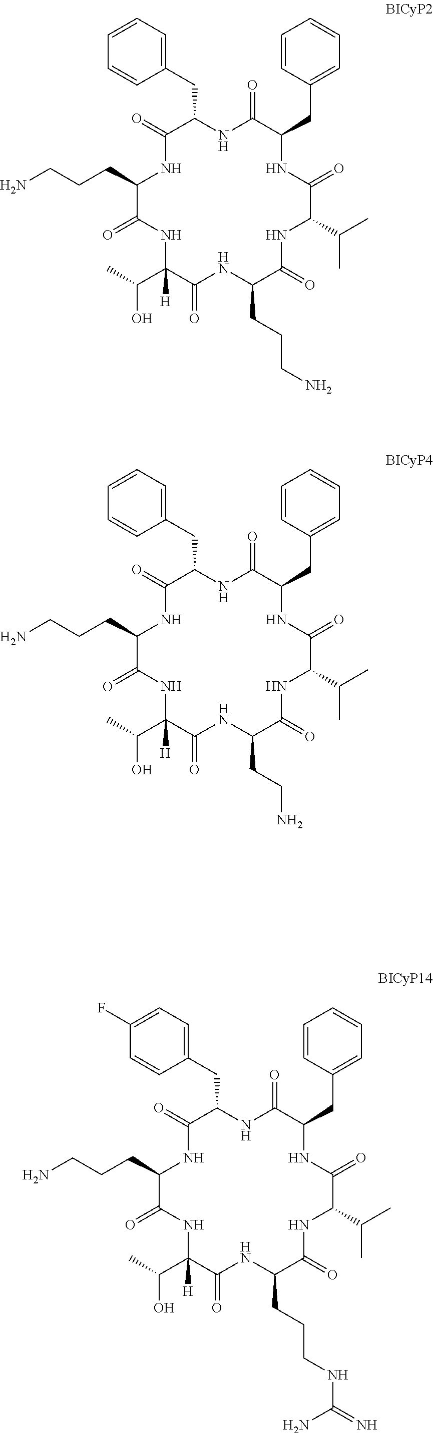 [SMILES: CC(C)[C@@H]1NC(=O)[C@@H](CCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(F)cc2)NC(=O)[C@@H](Cc2ccccc2)NC1=O]

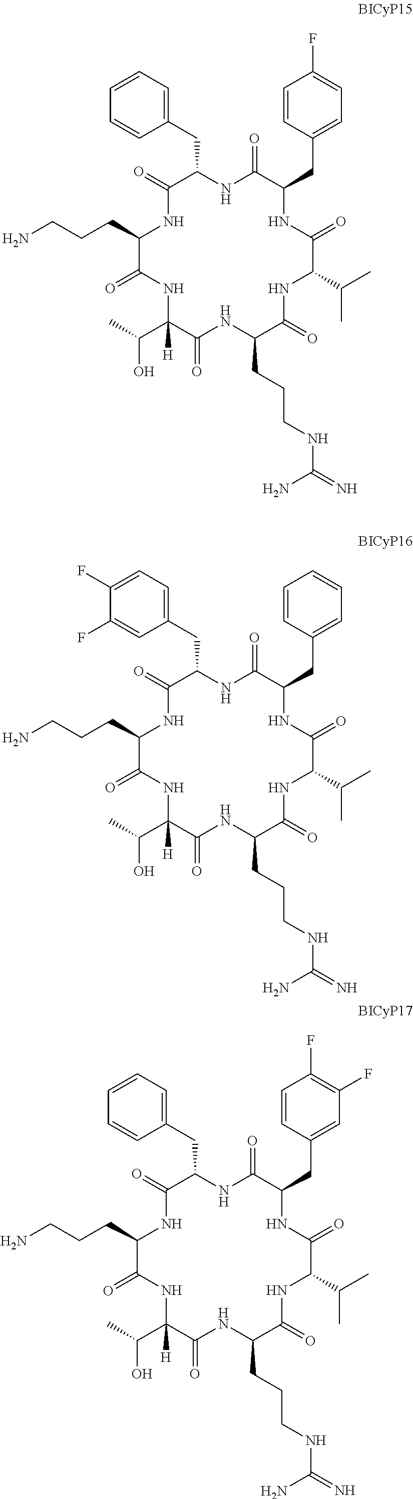 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(F)cc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(F)c(F)c2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(F)c(F)c2)NC1=O]

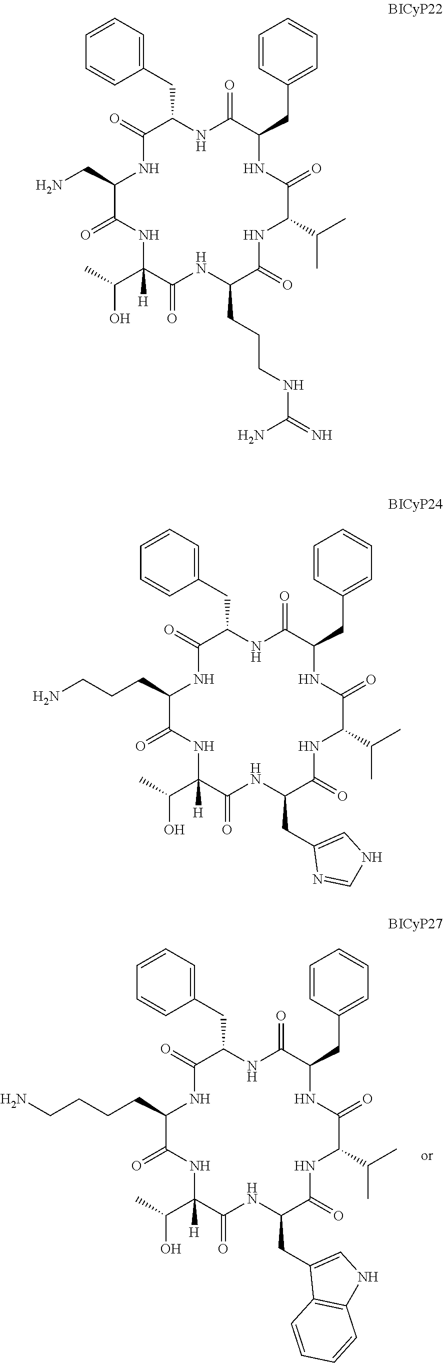 [SMILES: CC(C)[C@@H]1NC(=O)[C@@H](CCCNC(=N)N)NC(=O)[C@H]([C@@H](C)O)NC(=O)[C@@H](CN)NC(=O)[C@H](Cc2ccccc2)NC(=O)[C@@H](Cc2ccccc2)NC1=O ; CC(C)[C@@H]1NC(=O)[C@@H](Cc2c[nH]cn2)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](Cc2c[nH]c3ccccc23)NC(=O)[C@H]([C@@H](C)O)NC(=O)[C@@H](CCCCN)NC(=O)[C@H](Cc2ccccc2)NC(=O)[C@@H](Cc2ccccc2)NC1=O]

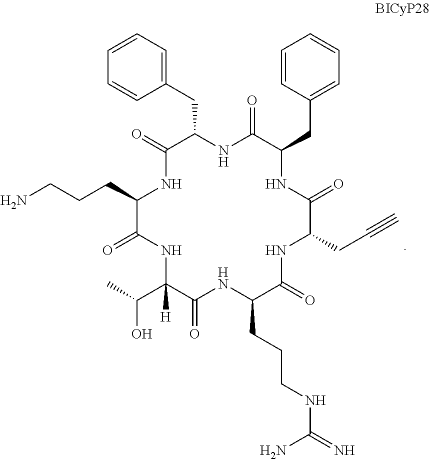 [SMILES: C#CC[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccccc2)NC1=O]

In some embodiments the cyclic peptide therapeutic agent pNP-51a comprises a formula

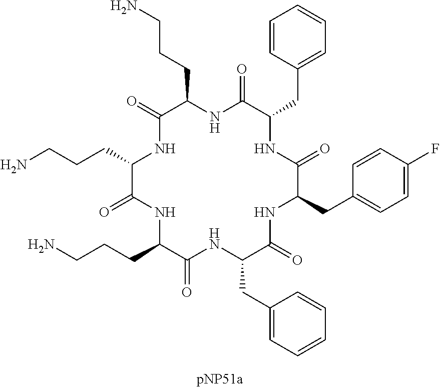 [SMILES: NCCC[C@@H]1NC(=O)[C@@H](CCCN)NC(=O)[C@H](Cc2ccccc2)NC(=O)[C@@H](Cc2ccc(F)cc2)NC(=O)[C@H](Cc2ccccc2)NC(=O)[C@@H](CCCN)NC1=O]

wherein AA1 is D-Orn, AA2 is Orn, AA3 is D-Orn, AA4 is Phe, AA5 is D-Phe (4F), and AA6 is Phe. The variants of pNP-51-a are: pNP-51b, wherein AA1 in pNP-51a formula is D-ala; pNP-51c, wherein AA1 in pNP-51a formula is D-lys; pNP-51d, wherein AA5 in pNP-51a formula is D-Phe (3, 4F); pNP-51e, wherein AA5 in pNP-51a formula is D-Phe (3F); or pNP-51f, wherein AA2 in pNP-51a formula is ala.

In some embodiments, the variants of BICyP3 provided are:

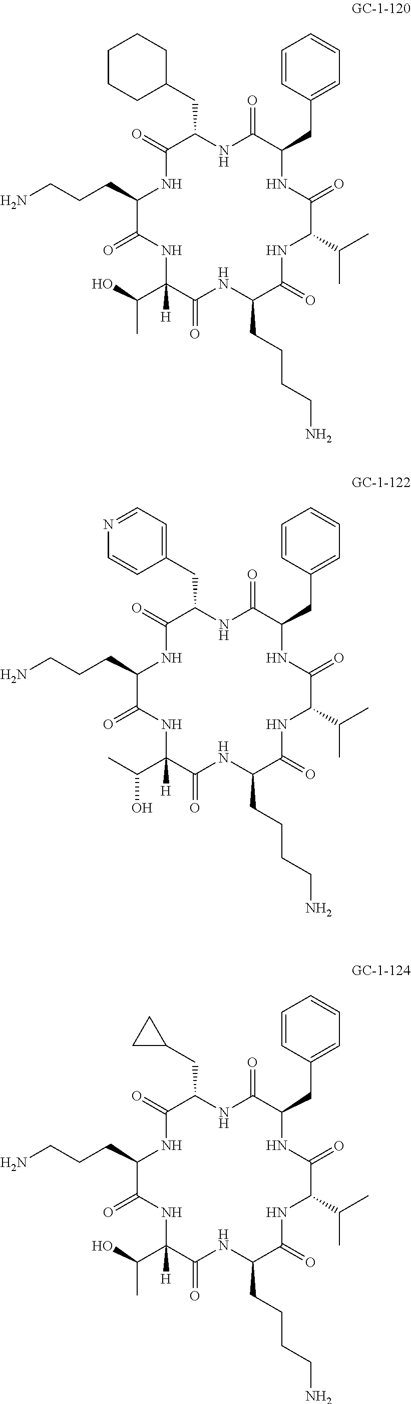 [SMILES: CC(C)[C@@H]1NC(=O)[C@@H](CCCCN)NC(=O)[C@H]([C@@H](C)O)NC(=O)[C@@H](CCCN)NC(=O)[C@H](CC2CCCCC2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccncc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](CC2CC2)NC(=O)[C@@H](Cc2ccccc2)NC1=O]

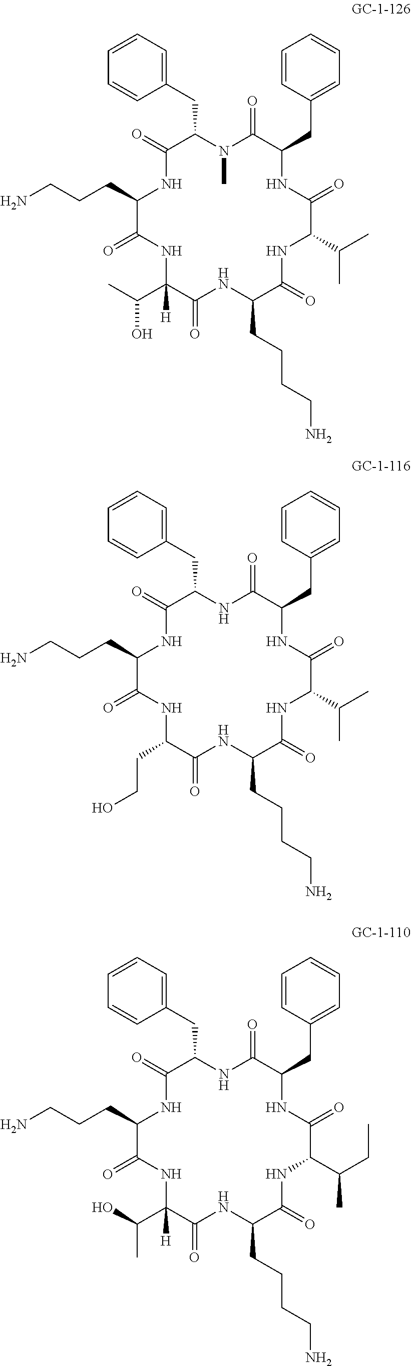

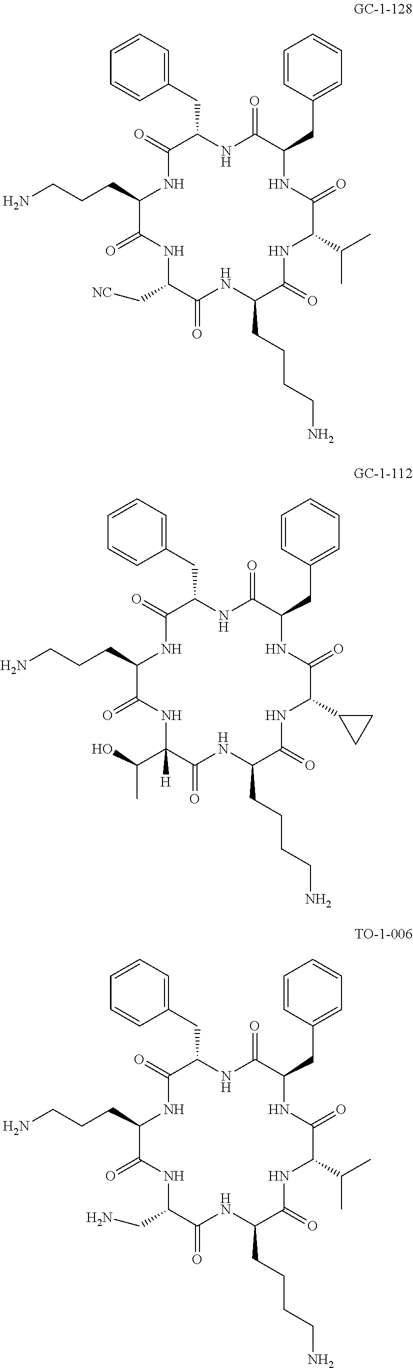 [SMILES: CC(C)[C@@H]1NC(=O)[C@@H](CCCCN)NC(=O)[C@H](CC#N)NC(=O)[C@@H](CCCN)NC(=O)[C@H](Cc2ccccc2)NC(=O)[C@@H](Cc2ccccc2)NC1=O ; C[C@@H](O)[C@@H]1NC(=O)[C@@H](CCCN)NC(=O)[C@H](Cc2ccccc2)NC(=O)[C@@H](Cc2ccccc2)NC(=O)[C@H](C2CC2)NC(=O)[C@@H](CCCCN)NC1=O ; CC(C)[C@@H]1NC(=O)[C@@H](CCCCN)NC(=O)[C@H](CN)NC(=O)[C@@H](CCCN)NC(=O)[C@H](Cc2ccccc2)NC(=O)[C@@H](Cc2ccccc2)NC1=O]

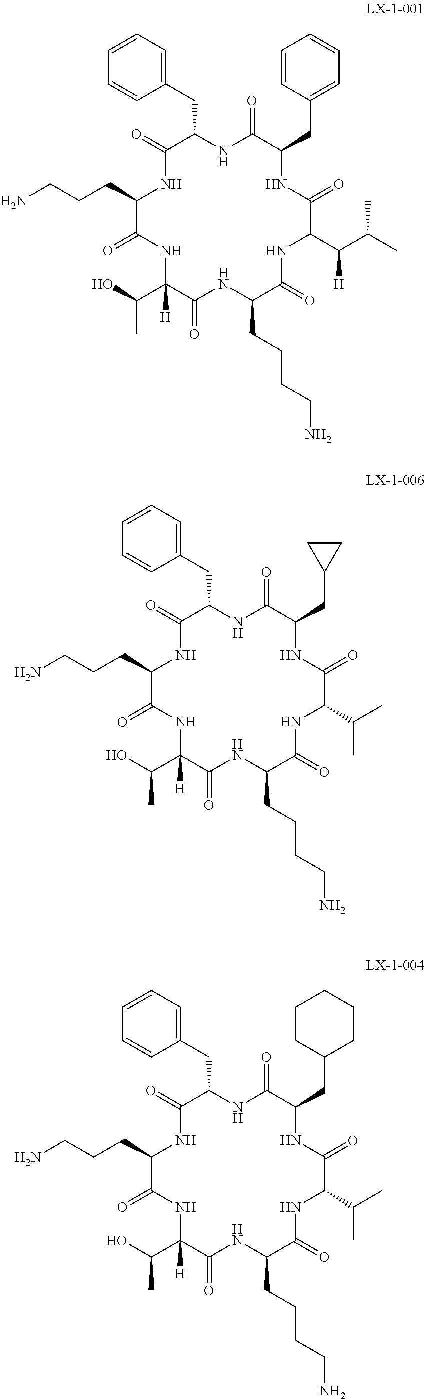

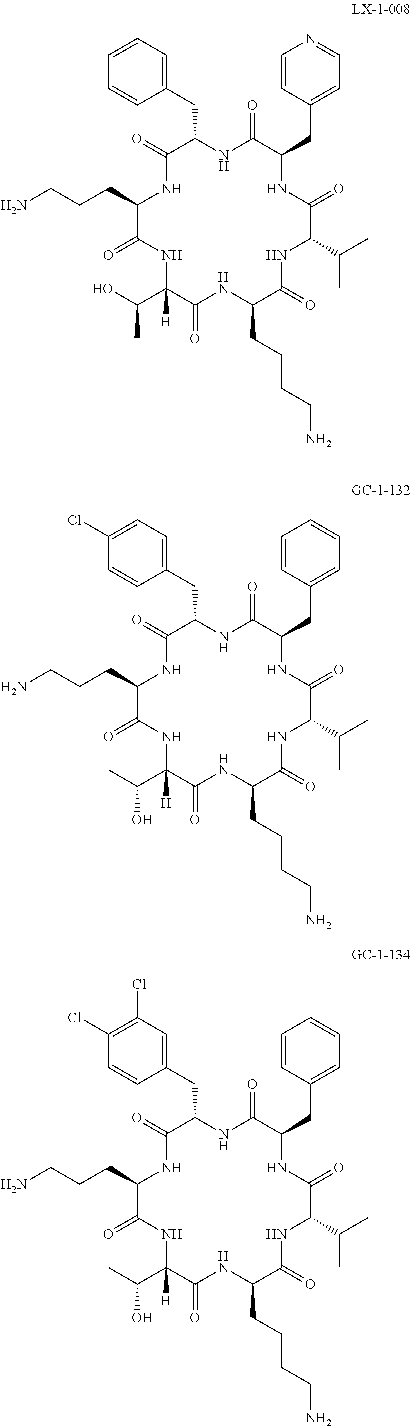 [SMILES: CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccncc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccc(Cl)cc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccc(Cl)c(Cl)c2)NC(=O)[C@@H](Cc2ccccc2)NC1=O]

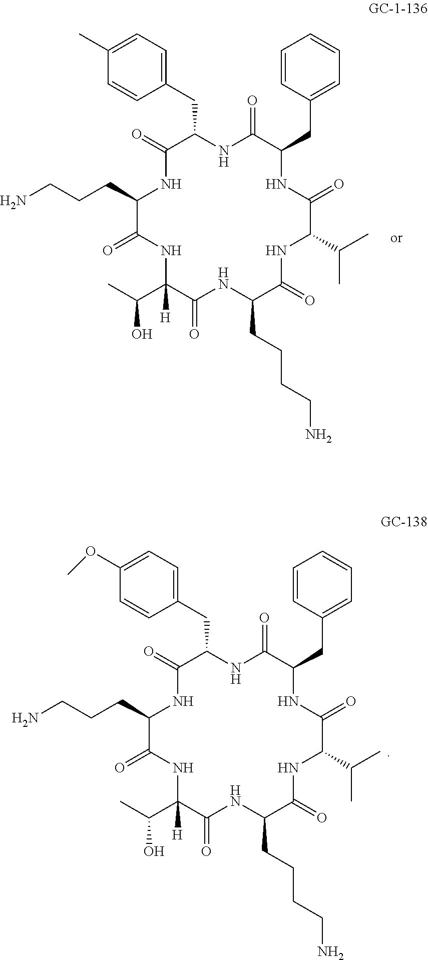 [SMILES: Cc1ccc(C[C@@H]2NC(=O)[C@@H](Cc3ccccc3)NC(=O)[C@H](C(C)C)NC(=O)[C@@H](CCCCN)NC(=O)[C@H]([C@H](C)O)NC(=O)[C@@H](CCCN)NC2=O)cc1 ; COc1ccc(C[C@@H]2NC(=O)[C@@H](Cc3ccccc3)NC(=O)[C@H](C(C)C)NC(=O)[C@@H](CCCCN)NC(=O)[C@H]([C@@H](C)O)NC(=O)[C@@H](CCCN)NC2=O)cc1]

In some embodiments, the BICyP3 and its variant can inhibit or treat infections or diseases caused by the pathogenic free-living amoeba. The pathogenic free-living amoeba can be a specific free-living amoeba, but not limited to Balamuthia mandrillaris (B. mandrillaris), Naegleria fowleri, and Acanthamoeba castellanii. Pathogenic free-living amoeba causes infection of the central nervous system and causes cutaneous and systemic diseases. Examples of the infections or diseases caused by these free-living amoeba include, but are not limited to, Balamuthia amoebic encephalitis (BAE), granulomatous amoebic encephalitis (GAE), primary amoebic meningoencephalitis (PAM), keratitis, lesions in skin and respiratory mucosa.

The term “amino acid” generally refers to an organic compound comprising both a carboxylic acid group and an amine group. The term “amino acid” includes both “natural” and “unnatural” or “non-natural” amino acids. Additionally, the term amino acid includes O-alkylated or N-alkylated amino acids, as well as amino acids having nitrogen or oxygen-containing side chains (such as Lys, Orn, or Ser) in which the nitrogen or oxygen atom has been acylated or alkylated. Amino acids may be pure L or D isomers or mixtures of L and D isomers, including racemic mixtures.

The term “natural amino acid” and equivalent expressions refer to L-amino acids commonly found in naturally occurring proteins. Examples of natural amino acids include, without limitation, alanine (Ala), cystein (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asp), proline (Pro), glutamine (Gin), arginine (Arg), serine (Ser), threonine (Thr), valine (Val), tryptophan (Trp), tyrosine (Tyr), β-alanine (β-ALA), and γ-aminobutyric acid (GABA).

The term “unnatural amino acid” refers to any derivative of a natural amino acid including D forms, and α- and β-amino acid derivatives. The terms “unnatural amino acid” and “non-natural amino acid” are used interchangeably herein and are meant to include the same moieties. It is noted that certain amino acids, e.g., hydroxyproline, that are classified as a non-natural amino acid herein, may be found in nature within a certain organism or a particular protein. Amino acids with many different protecting groups appropriate for immediate use in the solid-phase synthesis of peptides are commercially available.

The terms “inhibit,” “inhibiting,” “inhibited,” and “inhibition” with regard to inhibiting infection or disease caused by pathogenic free-living amoeba in a patient, such as a patient with BAE, refer to any degree of inhibition of one or more infections or diseases in the patient, wherein any degree of inhibition is beneficial to the patient. The term includes a method of inhibiting the onset of a disease or condition and/or its attendant symptoms or barring a subject from acquiring a disease. The term “inhibitory effect” or “inhibition” refers to the ability of a compound to reduce or block a specific biological or chemical activity, enzyme, or receptor, thereby preventing, suppressing, or altering its normal function.

The term “inhibitory amount” means any amount of a compound or a pharmaceutical composition comprising the compound that is sufficient to achieve an “inhibitor effect” or “inhibition”. The term “effective amount” or “therapeutically effective amount” means the amount of a compound that is effective to treat a disease or a disorder, such as metastatic cancer, at a reasonable benefit/risk ratio. The therapeutically effective amount of such compound will vary depending upon the patient and the disease or disorder being treated, the weight and age of the patient, the severity of the disease or disorder, the manner of administration, and the like, which can readily be determined by one of skill in the art.

The term “therapeutically effective amount” or “effective amount” refers to an amount of the active ingredient(s) that is(are) sufficient, when administered, to deliver efficaciously the active ingredient(s) for the treatment of a disease or condition of interest to a subject in need thereof. The therapeutically effective amount or effective amount of such composition will vary depending upon the patient and the disease or condition being treated, the weight and age of the patient, the severity of the disease or condition, the manner of administration, and the like, which can readily be determined by one of ordinary skill in the art.

For any compound, a therapeutically effective amount or effective amount can be initially determined from animal models. A therapeutically effective dose can also be determined from human data for compounds that have been tested in humans and for compounds that are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a prophylactic or therapeutically effective amount of a combination or composition described herein to a subject in need of such treatment. The treatment can be orientated symptomatically, for example, to suppress symptoms. It can be effected over a short period, be oriented over a medium term, or can be a long-term treatment, for example within the context of maintenance therapy.

Generally, daily oral doses of a compound are from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve the desired drug level, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

The compounds can be typically administered in admixture with a pharmaceutical carrier to give a pharmaceutical composition selected with regard to the intended route of administration and standard pharmaceutical practice. Pharmaceutical compositions can be formulated in a conventional manner using one or more physiologically acceptable carriers comprising excipients and/or auxiliaries that facilitate the processing of the compound. The pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a compound described herein is administered orally, the composition typically is in the form of a tablet, capsule, powder, solution, or elixir. When administered in tablet form, the composition additionally can contain a solid carrier, such as a gelatin or an adjuvant. The tablet, capsule, and powder can contain about 0.01% to about 95%, and preferably from about 1% to about 50%, of the combination of compounds. When administered in liquid form, a liquid carrier can be added, such as water, petroleum, or oils of animal or plant origin. The liquid form of the composition can further contain the physiological saline solution, dextrose or other saccharide solutions, or glycols. When administered in liquid form, the composition contains about 0.1% to about 90%, and preferably about 1% to about 50%, by weight, of the combination of compounds.

For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

The exact formulation, route of administration, and dosage of a pharmaceutical composition comprising an effective amount of the compound are determined by an individual physician in view of the diagnosed condition or disease. The dosage amount and interval can be adjusted individually to provide levels of the compound that are sufficient to maintain a prophylactic or therapeutic effect.

Toxicity and therapeutic efficacy of the combination can be determined by standard pharmaceutical procedures in cell cultures or experimental animals, e.g., for determining the maximum tolerated dose (MTD) of a compound, which is defined as the highest dose that causes no toxicity in animals. The therapeutic index is the dose ratio between the maximum tolerated dose and therapeutic effects (e.g., inhibition of tumor growth). The dosage can vary within this range depending upon the dosage form employed and the route of administration utilized. The determination of a therapeutically effective amount is well within the capability of those ordinarily skilled in the art, especially in light of the detailed disclosure provided herein.

A combination can be administered in an amount of about 0.005 to about 500 milligrams per dose, about 0.05 to about 250 milligrams per dose, or about 0.5 to about 100 milligrams per dose. For example, the combination can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

As stated above, a combination or composition described herein can be administered with one or more other prophylactically or therapeutically active agents.

It will be appreciated by persons skilled in the art that the present disclosure is not limited by what has been particularly shown and described herein above. Rather, the scope of the present disclosure includes both combinations and sub-combinations of the various features described hereinabove as well as variations and modifications that would occur to persons skilled in the art upon reading the specification and which are not in the prior art.

### Examples

The following examples serve to illustrate the present disclosure. The examples are not intended to limit the scope of the claimed invention in any way.

### Methods

All the methods are carried out in accordance with a previously reported method in ACS Chem. Biol., 2021, 16, 2604-2611, which is hereby specifically incorporated by reference for its teaching regarding the same.

Maintenance of Balamuthia mandrillaris

The pathogenic Balamuthia mandrillaris (CDC V039; ATCC 50209), a BAE strain, was isolated from a pregnant baboon at the San Diego zoo, USA, in 1986. Balamuthia mandrillaris trophozoites were routinely grown axenically at 37° C. in Balamuthia mandrillaris ITSON (BMI) medium (bacto casitone, 20 g/liter; Hanks' balanced salts, 68 mL/liter and 912 mL of deionized water). After autoclaving, 10% fetal bovine serum and 1% penicillin/streptomycin antibiotics were added to produce the complete BMI. All experiments were performed using logarithmic-phase trophozoites. Before experimentation, 0.25% trypsin-EDTA was used to detach the cells from the vented 75 cm² tissue culture flasks, and the cells were collected by centrifugation at 3214 RCF.

### In Vitro Cell Titer-Glo Trophocidal Assay

The trophocidal activity of cyclic peptides was assessed using the CellTiter-Glo 2.0® luminescent viability assay (Rice et al., Antimicrobial Agents and Chemotherapy, 2020, 64, 9, 476). In brief, all cyclic peptides were diluted in BMI and serially diluted in 2-fold dilutions six times with a final concentration of 10% dimethyl sulfoxide (DMSO) in clear 96-well plates (Costar 3370) “mother plates.” After serial dilutions were performed, 10 μL of peptides from the diluted stock plates were put into white 96-well plates, “assay-ready plates.” To combine parasites for screening, 90 μL of Balamuthia mandrillaris trophozoites were seeded at 16,000 cells/well from a stock concentration of 1.77×105/mL for a total time of 72 hours at 37° C. The peptide concentration was screened from 20 μM to 0.625 μM with a final DMSO (1%) screening concentration in the highest drug wells. DMSO (1%) and 90 μM of atorvastatin were used as the negative and positive control, respectively. At the 72-hour time point, 25 μL of CellTiter-Glo 2.0 Reagent® was added to all wells of the white 96-well plates. The plates were shaken at 250 rpm in a dark environment for 2 minutes to induce cell lysis, then incubated at room temperature for 10 min to stabilize the luminescent signal. The adenosine triphosphate (ATP) luminescent signal (relative light units; RLUs) was measured at 490 nm with a SpectraMax®i3X plate reader. Each peptide was tested in a minimum of three biological replicates using different populations of cells. Half maximal inhibitory concentration (IC₅₀) curves were generated using total ATP RLUs, where controls were calculated as the average of replicates using the Levenberg-Marquardt algorithm, using DMSO as the normalization control, as defined in CDD Vault.

### Mammalian Cytotoxicity Counter Screening

Cytotoxicity of the cyclic peptides was also determined by using the CellTiter-Glo 2.0® assay on A549 human lung carcinoma cells. A549 cells were seeded at a concentration of 1.6×10⁴ cells/mL in 96-well plates in the presence of serially diluted compounds, as described above. DMSO (1%) and 100 μM of chlorhexidine were used as the negative and positive controls. A549 were grown in F12K medium supplemented with 10% FBS and 1% penicillin/streptomycin antibiotics. The peptide concentration was screened from 100 μM to 3.125 μM. The total volume of each well was 100 μL, containing 10 μL compounds and 90 μL cells, and plates were incubated at 37° C., 5% CO₂ for 72 hours. At the 72-hours time point, CellTiter-Glo 2.0 Reagent® was added to all the wells and read by the SpectraMax® i3X plate reader as described above. Curve fitting using non-linear regression was carried out using the average of replicates and the Levenberg-Marquardt algorithm, using DMSO as the normalization control, as defined in CDD Vault. The selectivity index (SI) was calculated by using the equation: SI=(IC₅₀ of A549's)/(IC₅₀ of Balamuthia mandrillaris) to determine the specificity of inhibitors. A drug with a SI value ≥10 was considered the minimum standard for further evaluation as a hit drug.

### Statistical Analysis

The Z′ factor was used as a statistical measurement to assess the robustness of each drug plate. This factor uses the mean and standard deviation values of the positive and negative controls to assess data quality. The robustness of all of the plates screened had an excellent Z′-score value of 0.75 or above.

### Hemolysis Assay

100 μL of whole blood was combined with 500 μL of sterile 0.9% NaCl in a 1.5 mL Eppendorf tube, followed by thorough mixing through inversion. The resulting mixture underwent centrifugation at 500 rcf for 7 minutes, after which the supernatant was removed. The pellet was subjected to two additional washes, and following the final wash, 800 μL of red blood cell (RBC) buffer was added to resuspend the RBCs. Subsequently, 4 μL of the compounds (ranging from 100 to 0.78 μM in DMSO, final concentration), 76 μL RBC buffer, and 40 μL of resuspended RBC were added to a 96 U-well plate (VWR) and then incubated for 1 hour at 37° C. The plate underwent centrifugation at 500 g for 5 minutes, after which 75 μL of the supernatant was transferred to a 96 flat-well plate for absorbance measurement at 540 nm using a SpectraMax® iD3 plate reader. Percent hemolysis was determined based on the average absorbance of the positive (triton-X 100) and negative controls (DMSO), with a minimum of three biological triplicates conducted.

### Resin Loading

Resin loading was performed using a protocol reported in ACS Chem Biol, 2021, 16 (11), 2604-2611. 2-chlorotrityl chloride (2-CTC) resin (1 g, with a loading capacity of 0.56 mmol/g, mesh size 100-200), underwent swelling in dimethylformamide (DMF) for 5 minutes. After the removal of DMF, the swollen 2-CTC resin was exposed to fluorenylmethyloxycarbonyl (Fmoc)-protected amino acid (3 equiv.), N, N-diisopropylethylamine (DIPEA) (4 equiv.), and DMF (0.07 mM) for a duration of 2 hours. Subsequently, the solution was drained, and a mixture comprising of dichloromethane (DCM):methanol (MeOH):DIPEA in a ratio of 17:2:1 (0.07 mM) was employed to treat the resin, followed by agitation for 30 minutes to cap any remaining unreacted resin. After draining the solution, the resin underwent filtration and was washed thrice with DCM (5 mL each) and MeOH (5 mL each) before being dried for 1 hour. Resin loading was determined by exposing a small sample (1-3 mg) of the resin to piperidine-DMF (1:4) and determining the absorbance of the piperidine-dibenzofulvene adduct.

### Solid Phase Peptide Synthesis

Linear peptides were synthesized on a 0.05 mmol scale utilizing a PS3 peptide synthesizer (ACS Chem Biol, 2021, 16 (11), 2604-2611). Preloaded CTC resin, as detailed in the resin loading procedures above, was employed. Fmoc deprotection was carried out using a 20% piperidine/DMF solution (2×5 minutes). Dicyclohexylcarbodiimide (DIC), Oxyma Pure, and Fmoc-protected amino acids (each six equivalents) were utilized in a 1-hour coupling process. The coupling and deprotection steps were iterated until the desired linear peptide sequence was achieved.

### Peptide Cleavage

Following a reported method (ACS Chem Biol, 2021, 16 (11), 2604-2611), the peptide-linked resin obtained from solid phase peptide synthesis (SPPS) was swelled in DMF for 15 minutes. Following the draining of the DMF, the final Fmoc-protecting group was removed using a 20% piperidine/DMF solution for 15 minutes. The resin was then washed with DMF (3×2 mL) and DCM (3×2 mL) and dried. To confirm successful Fmoc deprotection, a Kaiser ninhydrin test was conducted. The deprotected peptide was cleaved from the resin using 25% HFIP/DCM for 30 minutes. The resultant solution was concentrated under vacuum, and the residue was resuspended in 50% H₂O/acetonitrile (MeCN), frozen, and lyophilized. The crude compound obtained was used without additional purification.

### Peptide Cyclization

Crude peptides (˜0.05 mmol), benzotriazolyloxy-tris[pyrrolidino]-phosphonium hexafluorophosphate (PyBop, 3 equivalents), DIPEA (6 equivalents), and DMF (1.25 mM) were subjected to overnight agitation (16-24 hours) and subsequently concentrated under vacuum. To the concentrated crude residue, 10 mL of 50% H₂O/MeCN was added, vortexed, and centrifuged to isolate the precipitated peptide. The resulting precipitate underwent additional washing with 10 mL of 50% H₂O/MeCN, followed by freezing and lyophilization. In case the residue did not precipitate, prep HPLC was employed to obtain the cyclized product.

### Global Deprotection

The crude cyclized product underwent treatment with trifluoroacetic acid (TFA):DCM:triisopropylsilyl (TIPS) (50:45: 5, 20 mM) for 2 hours. The volatiles were removed using air, and the peptides were precipitated with 2 mL of methyl tert-butyl ether (TBME). The precipitate was vortexed, centrifuged, washed with additional TBME, dissolved in H₂O:MeCN, frozen, and lyophilized.

### HPLC

High-performance liquid chromatography (HPLC) analysis and purification were conducted using an Agilent Technologies 1260 Infinity II preparative HPLC system. Purity analysis utilized a Luna C18 reverse phase column (5 μm, 150×4.6 mm, Phenomenex), while purification was carried out employing a Luna C18 reverse phase column (5 μm, 150×21.2 mm, Phenomenex). Eluent A consisted of H₂O with 0.1% formic acid (FA), while Eluent B comprised MeCN with 0.1% FA. The gradient for purity analysis was as follows: (A:B, flow rate 1 mL/min): 95:5, 0 min; 95:5, 1 min; 5:95, 20 min; 5:95, 25 min; 95:5, 30 min. For purification, the gradient was adjusted: (A:B, flow rate 20 mL/min): 95:5, 0 min; 95:5, 1 min; 5:95, 20 min; 5:95, 25 min; 95:5, 30 min.

### Result and Discussion

Exhibition of Significant Trophocidal Activity Against Balamuthia mandrillaris

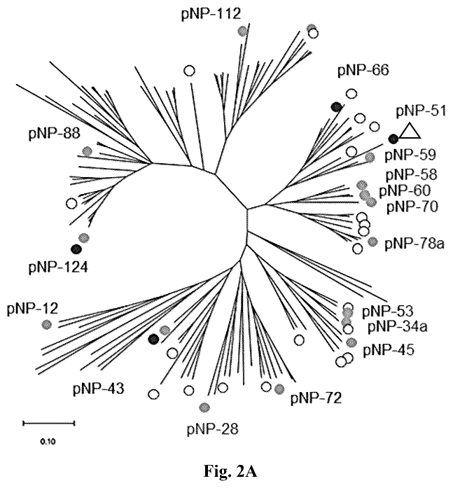

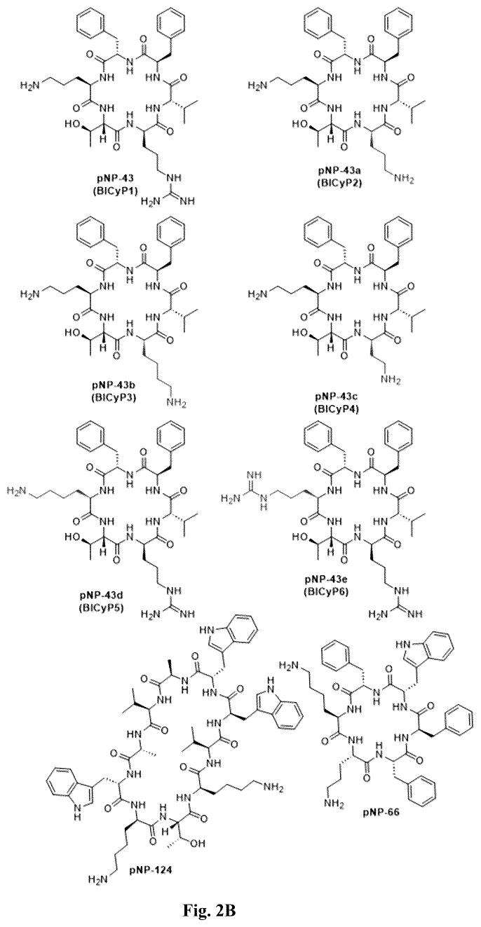

Balamuthia mandrillaris is a neglected infectious disease with a high mortality rate. Among the currently recommended drugs, only pentamidine has significant in vitro activity against Balamuthia mandrillaris, with a reported IC₅₀ of between 9 and 18 μM. To date, no one has shown curative in vivo activity due to the lack of robust translational pathobiological models. Recently, nitroxoline was screened against Balamuthia mandrillaris trophozoites in vitro and was identified to have an IC₅₀ of 4.8 μM (Laurie et al., mBio 2018, 9). Since the publication of these results, nitroxoline was successfully repurposed to treat a case of BAE disease in California, US (Spottiswoode et al., Emerg Infect Dis, 2023, 29, 197-201). The patient improved after nitroxoline administration and, excitingly, survived. However, kidney toxicity complications were observed. Given this dearth of effective, well-tolerated Balamuthia mandrillaris treatments and the great utility that cyclic peptides have shown as treatments for other diseases, forty-four cyclic peptides were screened from the SNaPP library at 16 μg/mL for trophocidal activity against Balamuthia mandrillaris, Acanthamoeba castellanii, and Naegleria fowleri. Active molecules were defined as those with greater than 50% reduction in growth. Eight active peptides for Balamuthia mandrillaris, sixteen active peptides for Acanthamoeba castellanii, and one active peptide for Naegleria fowleri were identified and validated via dose-response. The dose-response studies identified seven promising hits (IC₅₀≤10 μg) for Balamuthia mandrillaris, one hit for Acanthamoeba castellanii, and one hit for Naegleria fowleri, giving us a hit rate of 22%, 3%, and 3%, respectively (FIG. 2A). Of the identified hits, the most promising were BICyP1 for Balamuthia mandrillaris, pNP-51a for Acanthamoeba castellanii, and pNP-12 for Naegleria fowleri. BICyP1 was selected given its strong initial potency (IC₅₀=5 μM). Of the initial eight Balamuthia mandrillaris hits, five were closely structurally related to BICyP1, providing further support for pursuing this scaffold (FIG. 2B).

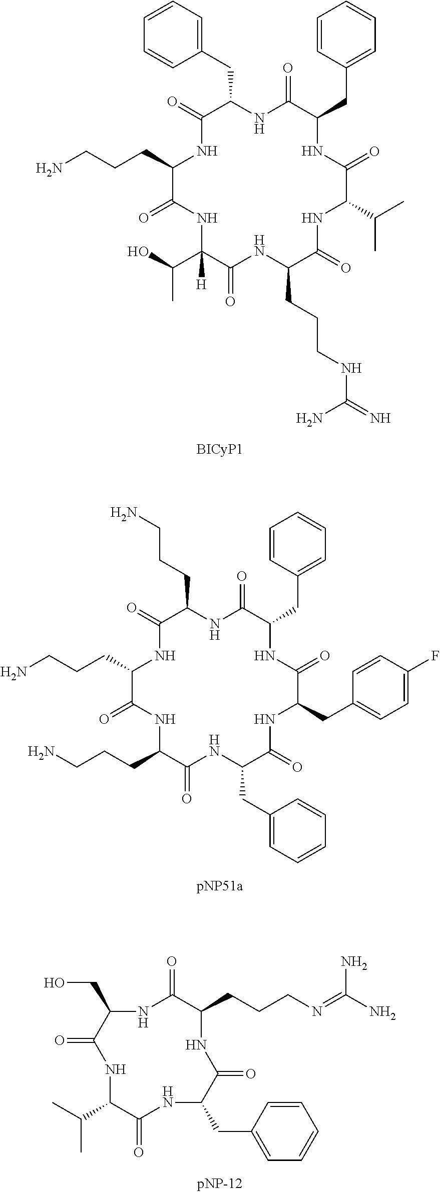

TABLE 1

| | IC₅₀ | IC₉₀ | |
|---|---|---|---|
| Name | (μg/mL) | (μg/mL) | |
| BICyP1 | 3.96 | 4.1 | Balamuthia |
| | | | mandrillaris |
| pNP-51a | 5.98 | >16.0 | Naegleria |
| | | | fowleri |
| pNP-12 | 5.27 | >16.0 | Acanthamoeba. |
| | | | castellanii |

An alanine scan was further performed for each position of the cyclic hexapeptide to identify residues that are necessary for activity (Table 2).

TABLE 2

| | Balamuthia. | | | |
|---|---|---|---|---|
| | mandrillaris | A549 | | Hemolysis |
| Name | IC₅₀ (μM) | IC₅₀ (μM) | SI | at 100 μM |
| BICyP1 | 5.0 ± 0.1 | >100 | >20 | <10% |
| BICyP3 | 3.8 ± 0.4 | >100 | >26 | <10% |
| BICyP5 | 4.1 ± 0.6 | 70.5 | 17 | <10% |
| BICyP7 | >20 | ND | ND | ND |
| BICyP8 | 11.9 ± 1.8 | 47.7 | 4.0 | <10% |
| BICyP9 | 8.5 ± 0.7 | >100 | >12 | <10% |
| BICyP10 | >20 | ND | ND | ND |
| BICyP11 | >20 | ND | ND | ND |
| BICyP12 | >20 | ND | ND | ND |
| BICyP13 | >20 | ND | ND | <10% |
| BICyP18 | 4.7 ± 1.0 | >100 | >21 | <10% |
| BICyP19 | 4.2 ± 0.8 | 64.4 | 15 | <10% |
| BICyP21 | 4.6 ± 0.2 | >100 | >22 | ND |
| BICyP26 | 3.9 ± 0.5 | 97.8 | 25 | <10% |

While all amino acids were found to contribute to the Balamuthia mandrillaris inhibitory activity, the D-orthinine (position 2 of BICyP1) and the threonine (position 3 of BICyP1) were found not to be essential (BICyP8 and BICyP9, respectively). A linear version of BICyP1 (BICyP13) was also tested, but it demonstrated a significant decrease in activity, confirming that the cyclic conformation is essential for activity. Following the alanine scan, further derivatization of BICyP1 was performed to identify more potent analogues. Modifying either of the phenylalanines (position 1 or 6 of BICyP1) with either a 4-flurophenylalanine (BICyP14 or BICyP15) or 3,4-flurophenylalanine (BICyP16 or BICyP17) resulted in a decrease in activity (Table 3). However, when a 3-flurophenylalanine (BICyP18 or BICyP19) was substituted at this position, the activity was maintained, suggesting that an electron-withdrawing group is tolerated in the meta position, but not the para position. Tyrosine substitution at the one position and six positions (BICyP20 and BICyP21) maintained activity.

TABLE 3
IC₅₀ of cyclic peptides against trophozoites
of Balamuthia mandrillaris in vitro.

| | B. mandrillaris | B. mandrillaris | A549 | |
|---|---|---|---|---|
| Compound | Mean IC₅₀ | Mean IC₉₀ | Mean IC₅₀ | Selectivity |
| Name | (μM) ±SD | (μM) ±SD | (μM) | Index (SI) |
| BICyP1 | 5.03 ± 0.06 | 5.73 ± 0.11 | >100 | >22 |
| BICyP3 | 3.79 ± 0.37 | 5.83 ± 0.33 | >100 | >26.39 |
| BICyP5 | 4.11 ± 0.56 | 5.66 ± 0.04 | 70.50 | 17.15 |
| BICyP18 | 4.68 ± 0.99 | 7.64 ± 1.94 | >100 | >21.37 |
| BICyP19 | 4.22 ± 0.77 | 5.89 ± 0.45 | 64.40 | 15.26 |
| BICyP20 | 5.41 ± 0.99 | 8.85 ± 1.62 | >100 | >18.48 |
| BICyP21 | 4.62 ± 0.17 | 7.38 ± 1.12 | >100 | >21.65 |
| BICyP25 | 6.25 ± 1.27 | 8.06 ± 5.28 | 49.00 | 7.84 |
| BICyP26 | 3.89 ± 0.54 | 6.65 ± 1.00 | 97.80 | 25.14 |
| pNP-51a | 3.50 ± 0.05 | 5.08 ± 0.12 | ND | ND |

ND—data not determined.

Explored by varying the chain length of nucleophilic amino acids at positions 2 and 4. First, D-arginine at position 4 was exchanged for an amino acid with a shorter chain length, such as D-lysine (BICyP3), D-ornithine (BICyP2), and D-diaminobutyric acid (BICyP4). While D-lysine (BICyP3) was well tolerated, the other modifications were not, suggesting that a side chain with 4 or more carbons at position 4 is important for activity. When the D-ornithine at position 2 was modified with D-lysine (BICyP5), resulting in a longer chain length, the inhibitory activity was maintained. However, shortening the chain with a D-diaminopropionic acid (BICyP22) and D-diaminobutyric acid (BICyP23) resulted in decreased activity. This suggests that a chain length of at least 3 carbons between the alpha-carbon and the basic residue is important for inhibitory activity. Interestingly, these results are similar to those previously found in the study of cationic quarternary ammonium compounds and alkylphosphocholines (Mooney et al., Sci Rep 2020, 10, 6420). While these molecules are quite structurally different from the cyclic peptides studied here, they also showed increased activity with increasing alkyl chain length. This points to the activity of these molecules being due, at least in part, to their ability to interact with the protist plasma membrane. This is consistent with one of the major mechanisms of antimicrobial peptides, interaction with the cell wall through hydrophobic or electrostatic interactions, leading to membrane damage or lysis, cytoplasmic leakage, and cell death.

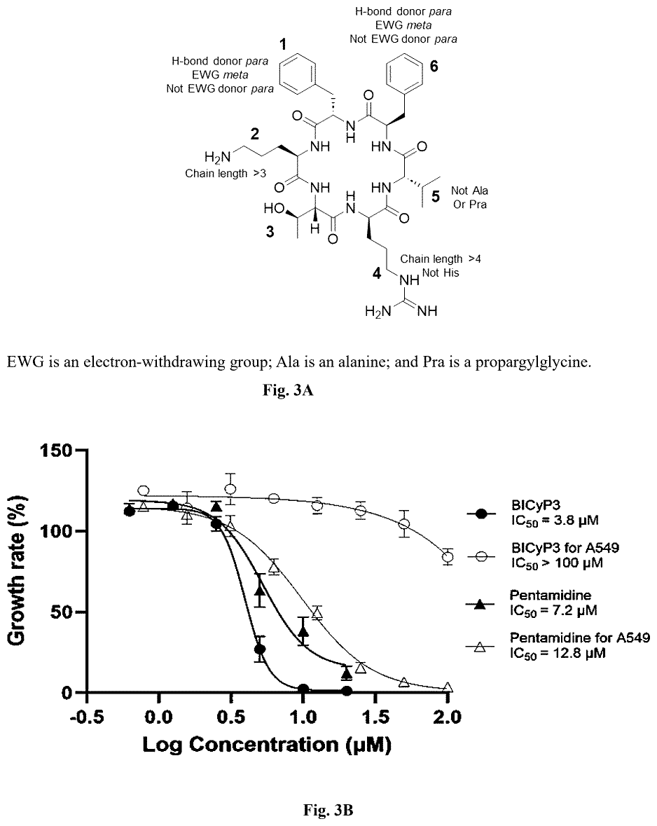

When position 4 is substituted with a D-histidine (BICyP24), activity decreases. However, when this modification is combined with position 2 being changed to a D-lysine (BICyP25) activity increases. For this reason, additional substitutions of position 4 were explored while keeping position 2 a D-lysine. Changing position 4 to D-lysine (BICyP26) maintains activity, while D-tryptophan (BICyP27) completely abolishes activity. This further confirms that a chain length greater than 4 is needed at position 4 and suggests that a locked conformation is not tolerated at that position. Given this information, a structure-activity relationship was determined (FIG. 3A). Excitingly, 6 compounds were identified with IC₅₀ₛ of less than 5 μM (BICyP3, BICyP5, BICyP18, BICyP19, BICyP21, and BICyP26). The top hit, BICyP3, was chosen to directly compare the in vitro performance against pentamidine. BICyP3 showed more potent efficacy with the IC₅₀ of 2.43 μM, compared to the IC₅₀ of 6.59 μM for pentamidine (FIG. 3B).

The ability of these molecules to inhibit the growth of other FLA was screened. Interestingly, neither our initial hit (BICyP1) nor the derivatives tested showed strong activity (IC₅₀<5 μM). The selectivity of these cyclic peptides for Balamuthia mandrillaris over Acanthamoeba and Naegleria is quite interesting and points to a unique mechanism of action. It is possible that this selectivity may be due to differences in the composition of plasma membranes, given that many cyclic peptides act at the plasma membrane.

Table 4 illustrates IC₅₀ values of variants of BICyP3.

TABLE 4
IC₅₀ of variants of BICyP3 against trophozoites
of Balamuthia mandrillaris in vitro

| Compound | B. mandrillaris |
|---|---|
| Name | IC₅₀ (μM) |
| BICyP3 | 3.79 ± 0.37 |
| GC-1-120 | 4.45 |
| TO-1-006 | 5.48 |
| GC-1-116 | 4.69 |
| GC-1-128 | 3.34 |
| LX-1-001 | 6.84 |
| GC-1-110 | 5.66 |
| LX-1-004 | 4.76 |

### Exhibition of Minimal Toxicity Against Human Cells

Many of the currently used medicines for Balamuthia mandrillaris have severe toxicities. For example, a 57-year-old cutaneous Balamuthia mandrillaris infected patient died with severe liver damage, potentially due to complications of the diminazene aceturate treatment they received (Wang et al., Clin Infect Dis, 2022, 75, 1637-1640). Another patient with Balamuthia mandrillaris GAE was treated for more than 2 months with the recommended multiple-drug regimen (flucytosine, pentamidine, fluconazole, sulfadiazine, azithromycin, miltefosine, and albendazole) and developed adverse effects, including acute kidney injury and myelosuppression (Haston et al., Curr Opin Infect Dis, 2023, 36, 186-191). These examples indicate that new treatments with lower toxicities are needed for Balamuthia mandrillaris. Cyclic peptide natural products are crucial sources of drug candidates because of their wide range of activities. Compared with linear peptides, the circular structures enable higher rigidity of cyclic peptides to reduce the entropic cost of the receptor binding that provides higher affinity, specificity, and selectivity to the target, which results in lower to minimal cytotoxicity. Given the promising activity of several cyclic peptides against Balamuthia mandrillaris, evaluated the most promising compounds (IC₅₀<5 μM) for their selectivity. Specifically, their cytotoxicity against a human cell line (A549) and their hemolytic activity (Table 2, FIG. 3B) were examined. Gratifyingly, all hit compounds tested showed minimal toxicity to A549 lung carcinoma cells (IC₅₀≥64 μM). The selectivity index (SI=A549 IC₅₀/Balamuthia mandrillaris IC₅₀) was determined for each compound, with the lead compounds having SI≥15. The cytotoxicity of pentamidine on A549 (IC₅₀=12.8 μM) was tested, demonstrating a higher cytotoxicity value and a poor SI of 1.78. Additionally, none of the compounds caused hemolysis at the highest concentration tested (100 μM). Overall, this data suggests that cyclic peptides are likely to have minimal to no cellular cytotoxicity to humans and and have selectivity at inhibiting Balamuthia mandrillaris.

Overall, inhibitors of Balamuthia mandrillaris were identified. The initial screen revealed 8 cyclic peptides with an IC₅₀ less 16 μg/mL. Derivatives were then synthesized, enabling the development of a robust structural activity relationship. The multiple derivatives were identified to have an IC₅₀ of less than 5 μM and an IC₉₀ of less than 10 μM, with minimal to no toxicity to mammalian cells. Given that pentamidine, one of the current gold standards of Balamuthia mandrillaris treatment, has a lower efficacy against Balamuthia mandrillaris with an IC₅₀ of 7.2 μM, higher cytotoxicity, and poor selectivity, these cyclic peptides show great promise as leads for challenging to-treat disease.

While specific embodiments of the subject invention have been disclosed herein, the above specification is illustrative and not restrictive. While this invention has been particularly shown and described with references to preferred embodiments thereof, it will be understood by those skilled in the art that various changes in form and details may be made therein without departing from the scope of the invention encompassed by the appended claims. Many variations of the invention will become apparent to those of skilled art upon review of this specification. Unless otherwise indicated, all numbers expressing reaction conditions, quantities of ingredients, and so forth, as used in this specification, are to be understood as being modified in all instances by the term “about” Accordingly, unless indicated to the contrary, the numerical parameters set forth herein are approximations that can vary depending upon the desired properties sought to be obtained by the present disclosure.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid the reading of the document and is not to be interpreted as limiting. Further, information that is relevant to a section heading may occur within or outside of that particular section. The terms “including” and “having” are defined as comprising (i.e., open language).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

What is claimed is:

1. A cyclic peptide therapeutic agent selected from: 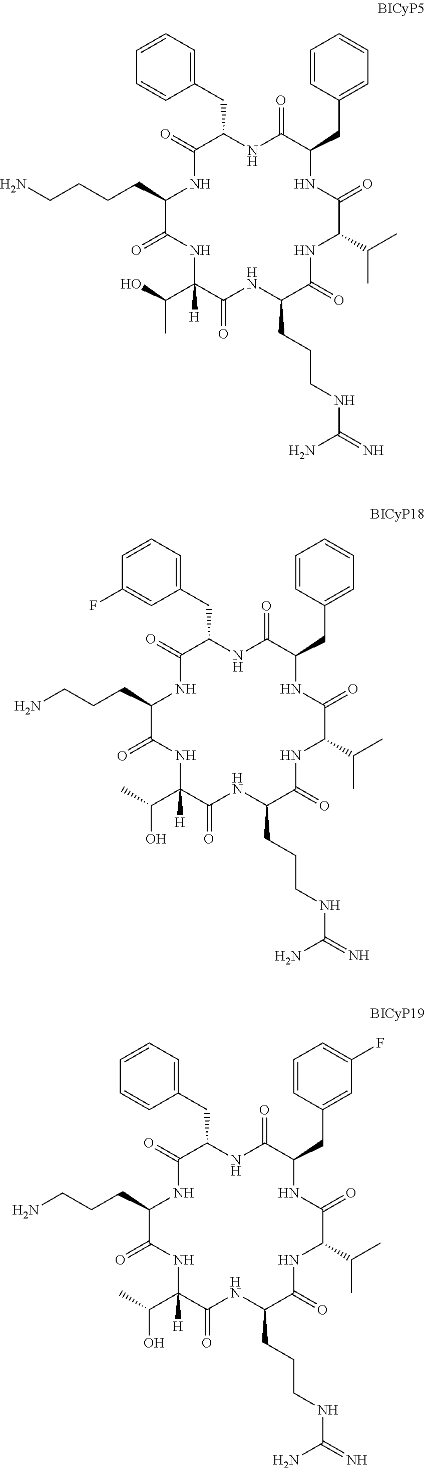 [SMILES: CC(C)[C@@H]1NC(=O)[C@@H](CCCNC(=N)N)NC(=O)[C@H]([C@@H](C)O)NC(=O)[C@@H](CCCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2cccc(F)c2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2cccc(F)c2)NC1=O] 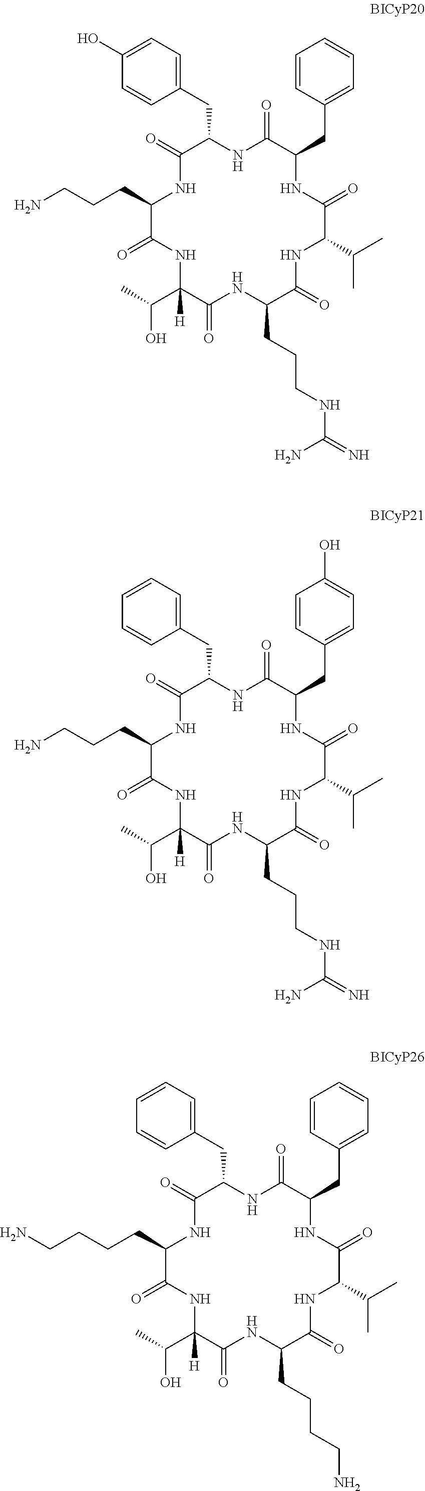 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(O)cc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(O)cc2)NC1=O ; CC(C)[C@@H]1NC(=O)[C@@H](CCCCN)NC(=O)[C@H]([C@@H](C)O)NC(=O)[C@@H](CCCCN)NC(=O)[C@H](Cc2ccccc2)NC(=O)[C@@H](Cc2ccccc2)NC1=O] 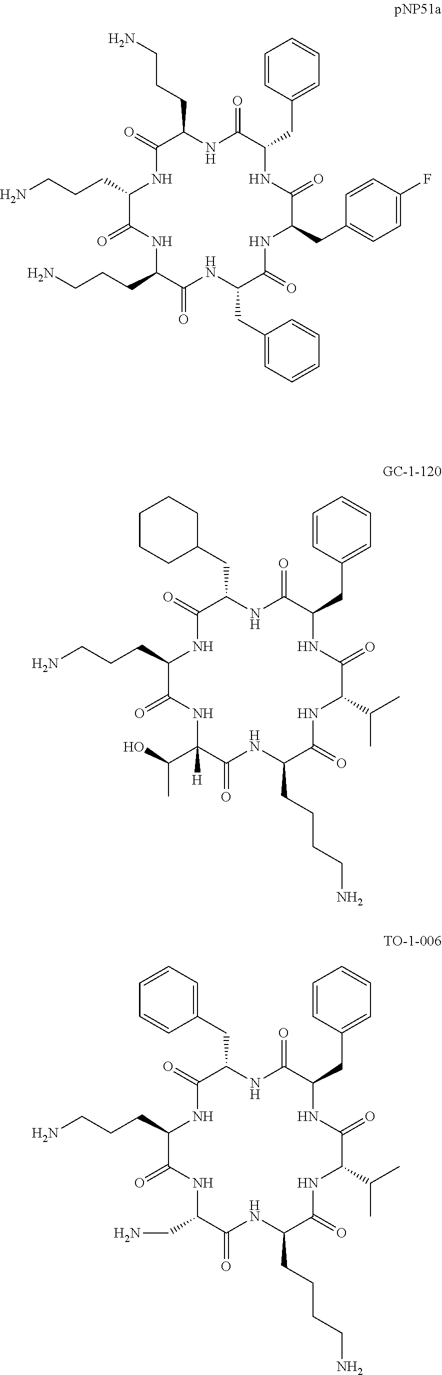 [SMILES: NCCC[C@@H]1NC(=O)[C@@H](CCCN)NC(=O)[C@H](Cc2ccccc2)NC(=O)[C@@H](Cc2ccc(F)cc2)NC(=O)[C@H](Cc2ccccc2)NC(=O)[C@@H](CCCN)NC1=O ; CC(C)[C@@H]1NC(=O)[C@@H](CCCCN)NC(=O)[C@H]([C@@H](C)O)NC(=O)[C@@H](CCCN)NC(=O)[C@H](CC2CCCCC2)NC(=O)[C@@H](Cc2ccccc2)NC1=O ; CC(C)[C@@H]1NC(=O)[C@@H](CCCCN)NC(=O)[C@H](CN)NC(=O)[C@@H](CCCN)NC(=O)[C@H](Cc2ccccc2)NC(=O)[C@@H](Cc2ccccc2)NC1=O] 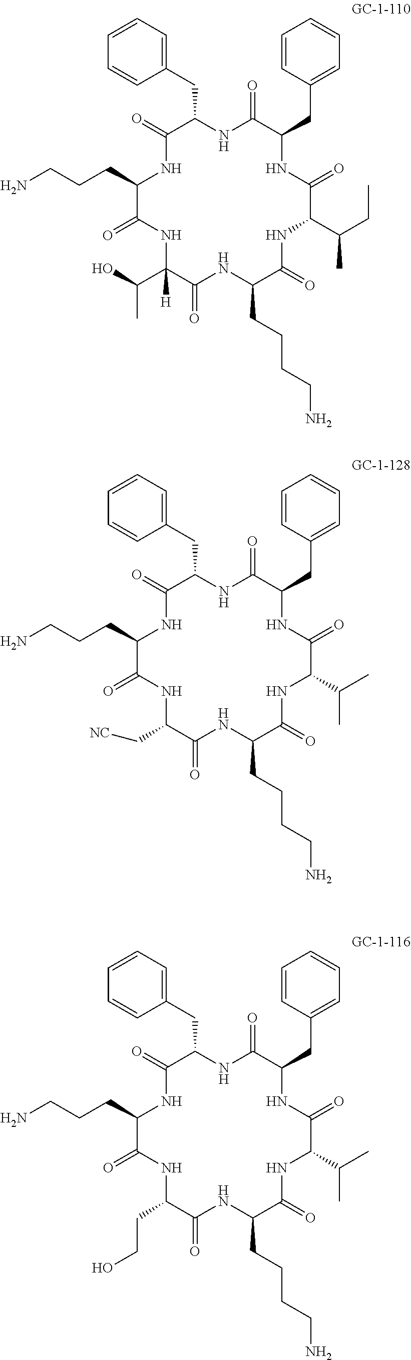 [SMILES: CC[C@@H](C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H](CC#N)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H](CCO)NC(=O)[C@@H](CCCN)NC(=O)[C@H](Cc2ccccc2)NC(=O)[C@@H](Cc2ccccc2)NC1=O] 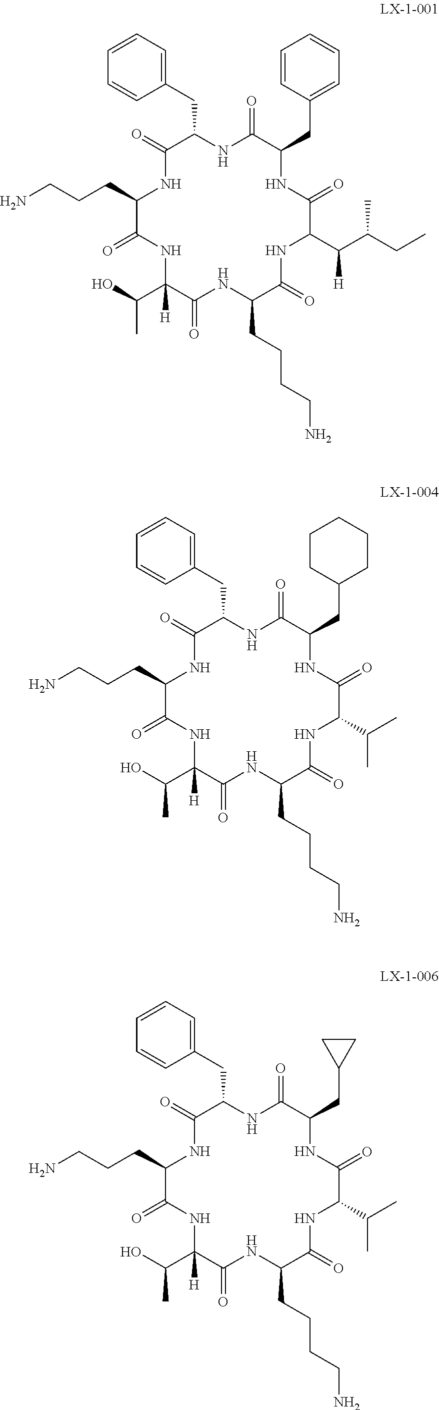 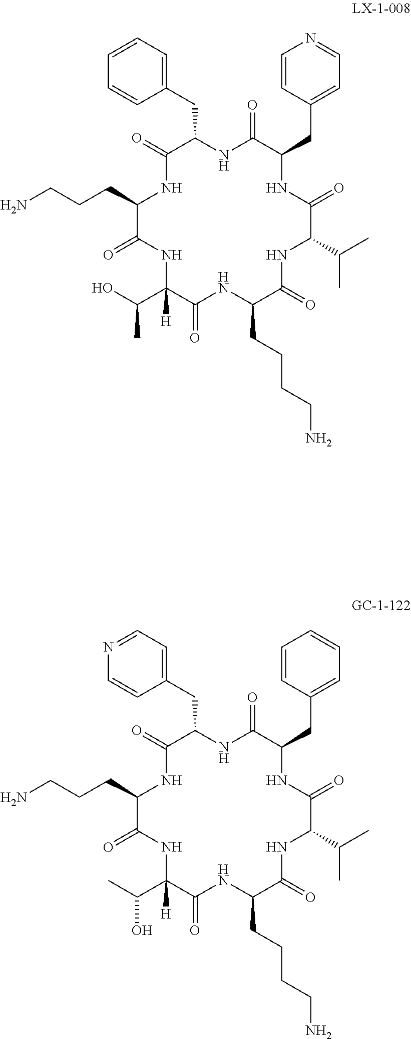 [SMILES: CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccncc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccncc2)NC(=O)[C@@H](Cc2ccccc2)NC1=O] 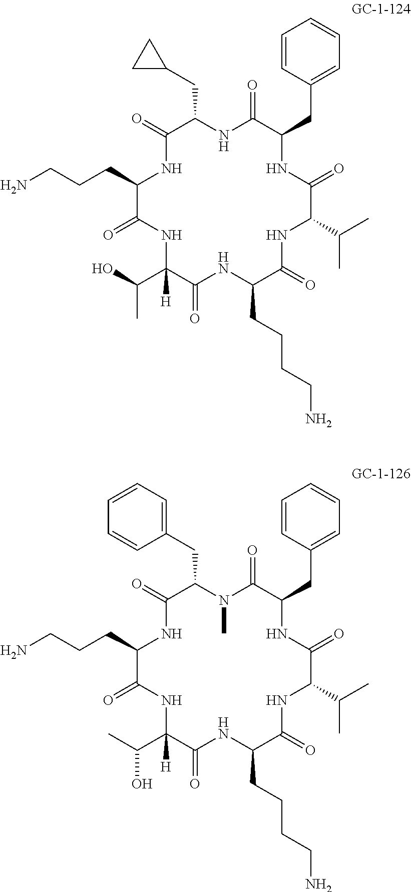 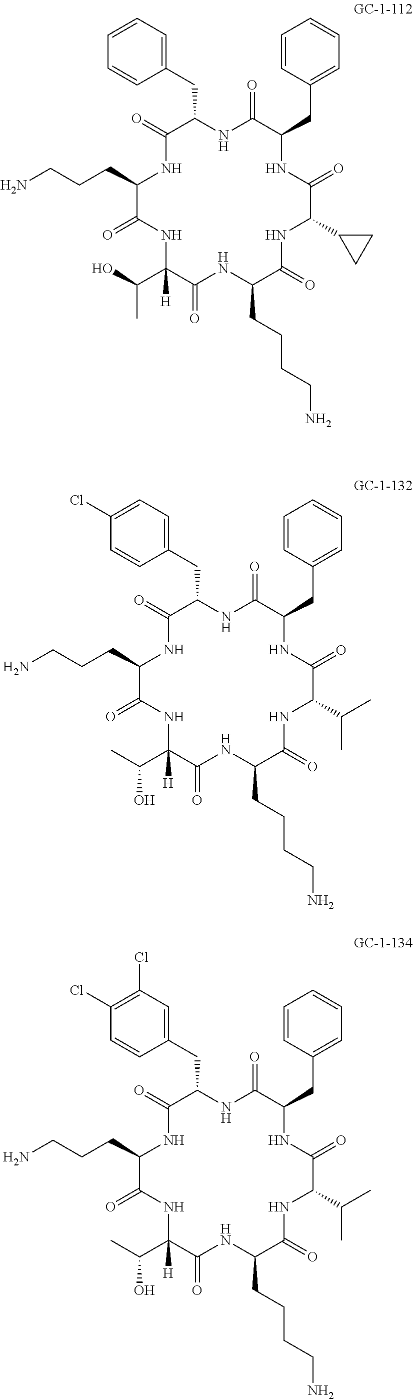 [SMILES: C[C@@H](O)[C@@H]1NC(=O)[C@@H](CCCN)NC(=O)[C@H](Cc2ccccc2)NC(=O)[C@@H](Cc2ccccc2)NC(=O)[C@H](C2CC2)NC(=O)[C@@H](CCCCN)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccc(Cl)cc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccc(Cl)c(Cl)c2)NC(=O)[C@@H](Cc2ccccc2)NC1=O] 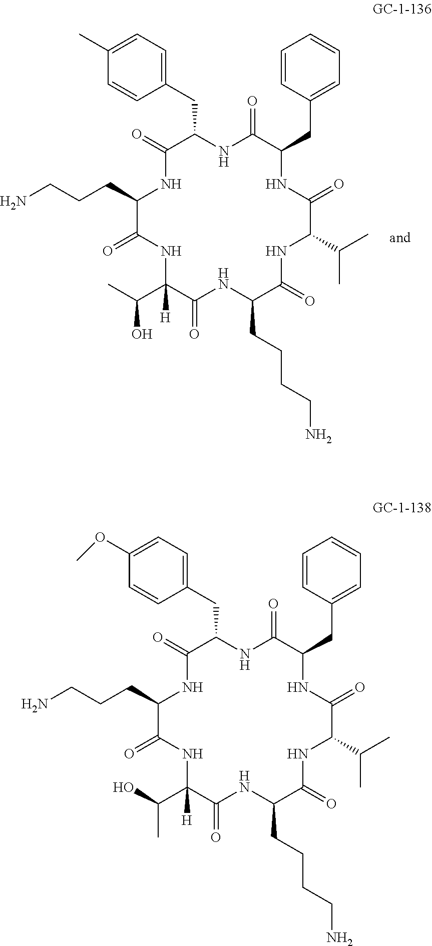 [SMILES: Cc1ccc(C[C@@H]2NC(=O)[C@@H](Cc3ccccc3)NC(=O)[C@H](C(C)C)NC(=O)[C@@H](CCCCN)NC(=O)[C@H]([C@H](C)O)NC(=O)[C@@H](CCCN)NC2=O)cc1 ; COc1ccc(C[C@@H]2NC(=O)[C@@H](Cc3ccccc3)NC(=O)[C@H](C(C)C)NC(=O)[C@@H](CCCCN)NC(=O)[C@H]([C@@H](C)O)NC(=O)[C@@H](CCCN)NC2=O)cc1]
or a variant thereof.

2. The pharmaceutical composition comprising a cyclic peptide therapeutic agent of claim 1 and a pharmaceutically acceptable carrier, diluent, or excipient.

3. The pharmaceutical composition of claim 2, further comprising one or more additional therapeutic agents, one or more of which can be other than a cyclic peptide therapeutic agent.

4. A method of inhibiting an infection or a disease caused by a pathogenic free-living amoeba in a patient in need thereof, which method comprises administering an inhibitory effective amount of a cyclic peptide therapeutic agent selected from 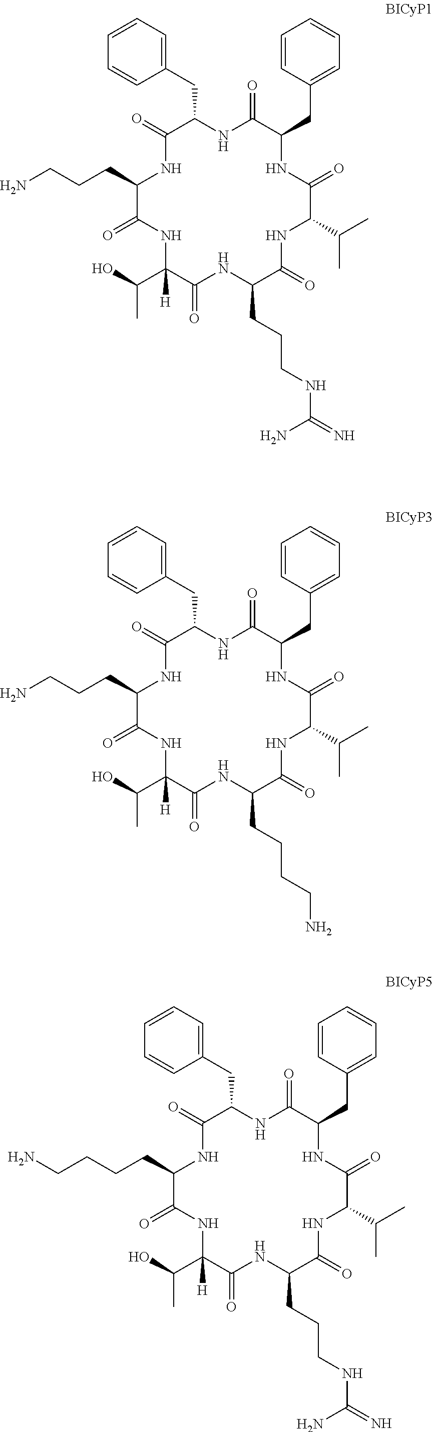 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCCN)NC(=O)[C@H](Cc2ccccc2)NC(=O)[C@@H](Cc2ccccc2)NC1=O] 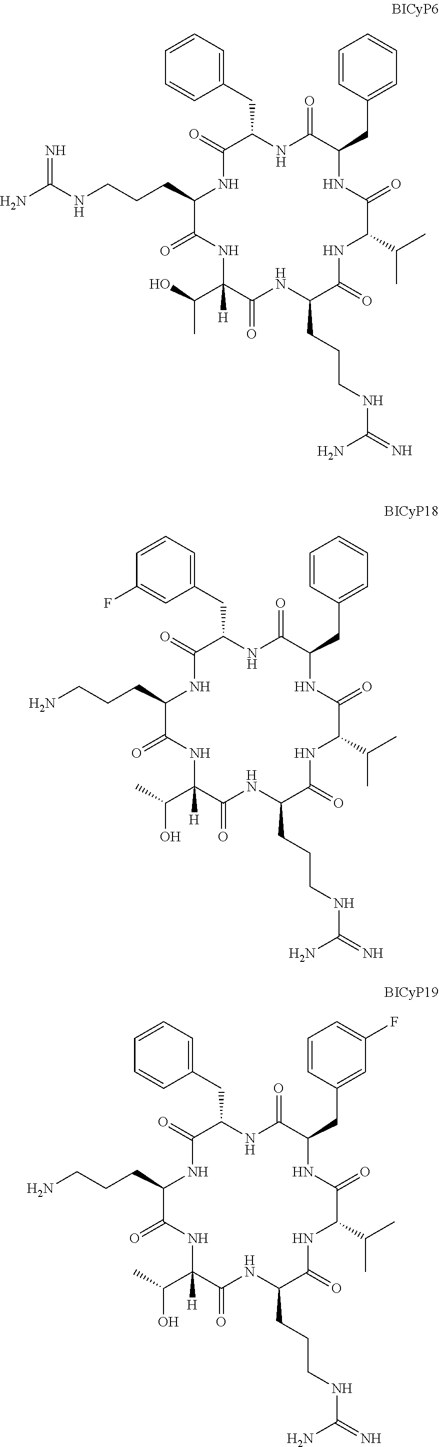 [SMILES: CC(C)[C@@H]1NC(=O)[C@@H](CCCNC(=N)N)NC(=O)[C@H]([C@@H](C)O)NC(=O)[C@@H](CCCNC(=N)N)NC(=O)[C@H](Cc2ccccc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2cccc(F)c2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2cccc(F)c2)NC1=O] 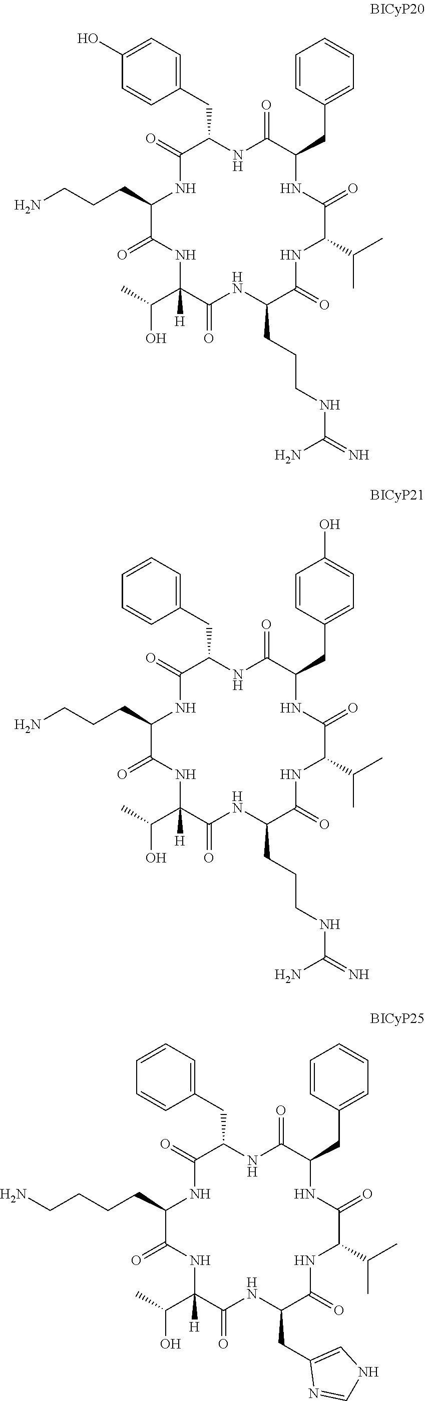 [SMILES: CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccc(O)cc2)NC(=O)[C@@H](Cc2ccccc2)NC1=O ; CC(C)[C@@H]1NC(=O)[C@@H](CCCNC(=N)N)NC(=O)[C@H]([C@@H](C)O)NC(=O)[C@@H](CCCN)NC(=O)[C@H](Cc2ccccc2)NC(=O)[C@@H](Cc2ccc(O)cc2)NC1=O ; CC(C)[C@@H]1NC(=O)[C@@H](Cc2c[nH]cn2)NC(=O)[C@H]([C@@H](C)O)NC(=O)[C@@H](CCCCN)NC(=O)[C@H](Cc2ccccc2)NC(=O)[C@@H](Cc2ccccc2)NC1=O] 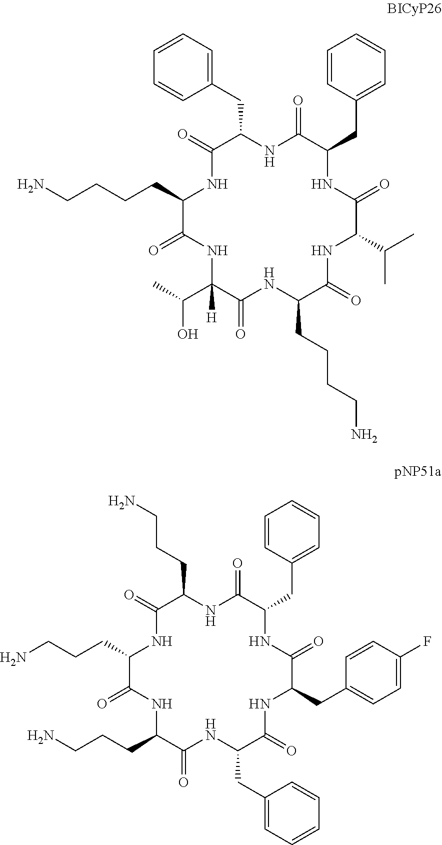 [SMILES: CC(C)[C@@H]1NC(=O)[C@@H](CCCCN)NC(=O)[C@H]([C@@H](C)O)NC(=O)[C@@H](CCCCN)NC(=O)[C@H](Cc2ccccc2)NC(=O)[C@@H](Cc2ccccc2)NC1=O ; NCCC[C@@H]1NC(=O)[C@@H](CCCN)NC(=O)[C@H](Cc2ccccc2)NC(=O)[C@@H](Cc2ccc(F)cc2)NC(=O)[C@H](Cc2ccccc2)NC(=O)[C@@H](CCCN)NC1=O] 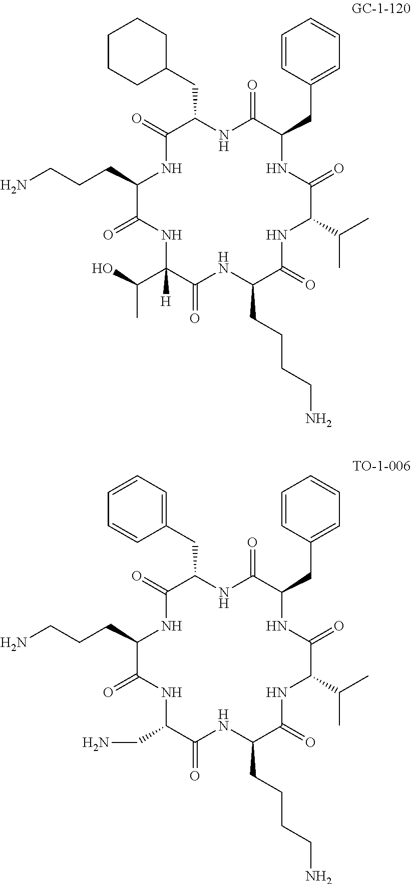 [SMILES: CC(C)[C@@H]1NC(=O)[C@@H](CCCCN)NC(=O)[C@H]([C@@H](C)O)NC(=O)[C@@H](CCCN)NC(=O)[C@H](CC2CCCCC2)NC(=O)[C@@H](Cc2ccccc2)NC1=O ; CC(C)[C@@H]1NC(=O)[C@@H](CCCCN)NC(=O)[C@H](CN)NC(=O)[C@@H](CCCN)NC(=O)[C@H](Cc2ccccc2)NC(=O)[C@@H](Cc2ccccc2)NC1=O] 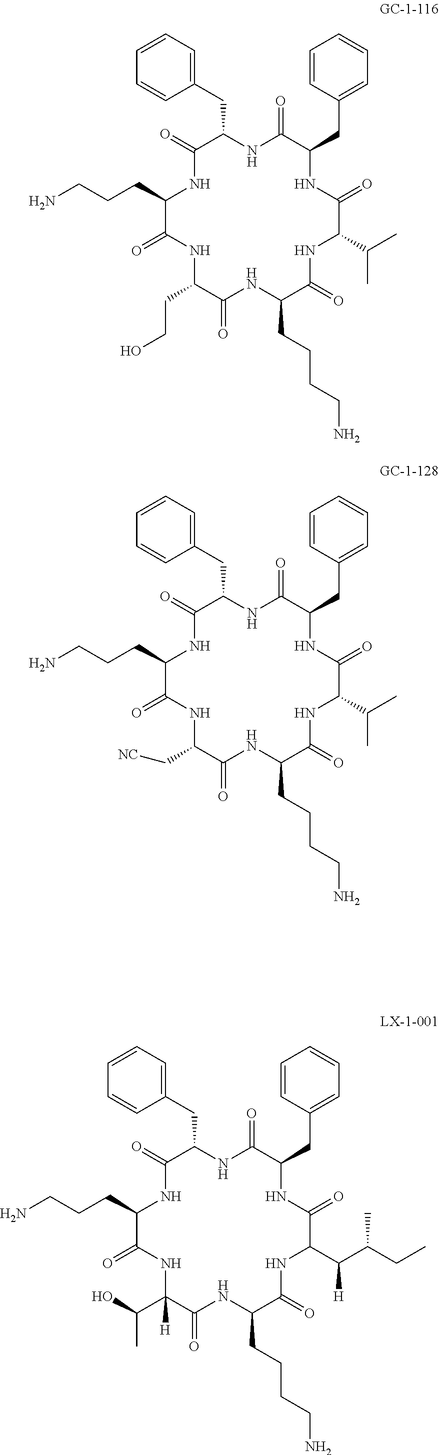 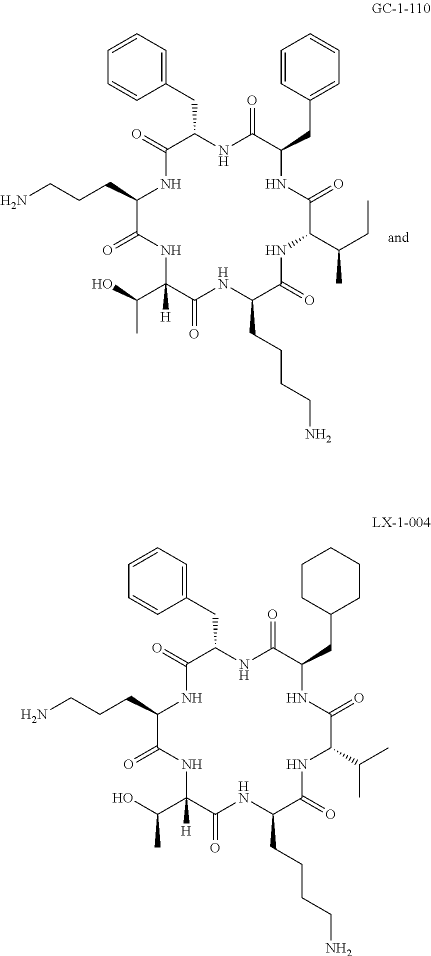 [SMILES: CC[C@@H](C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](Cc2ccccc2)NC1=O ; CC(C)[C@@H]1NC(=O)[C@@H](CCCCN)NC(=O)[C@H]([C@@H](C)O)NC(=O)[C@@H](CCCN)NC(=O)[C@H](Cc2ccccc2)NC(=O)[C@@H](CC2CCCCC2)NC1=O]
or a variant thereof or a pharmaceutical composition comprising the same, whereupon the infection or disease caused by the pathogenic free-living amoeba in the patient is inhibited.

5. The method of claim 4, wherein the pathogenic free-living amoeba is Balamuthia mandrillaris.

6. The method of claim 4, wherein the pathogenic free-living amoeba is Naegleria fowleri.

7. The method of claim 4, wherein the pathogenic free-living amoeba is Acanthamoeba castellanii.
